(19) United States
(12) Patent Application Publication
(10) Pub. No.: US 2026/0117222 A1
(43) Pub. Date: Apr. 30, 2026

(54) NON-INVASIVE, STOOL-DERIVED mRNA-BASED METHOD FOR ASSESSING GUT HEALTH AND DEVELOPMENT

(71) Applicant: The Texas A&M University System, College Station, TX (US)

(72) Inventors: Robert S. Chapkin, College Station, TX (US); Laurie Davidson Chapkin, College Station, TX (US)

(21) Appl. No.: 19/366,047

(22) Filed: Oct. 22, 2025

Related U.S. Application Data
(60) Provisional application No. 63/714,222, filed on Oct. 31, 2024.

(51) Int. Cl. C12N 15/10; G16B 25/10; G16H 20/60

(52) U.S. Cl. CPC C12N 15/1093; G16B 25/10; G16H 20/60

(57) ABSTRACT

The present disclosure relates to methods and compositions for producing a sequencing library from mRNA in a stool sample. The present disclosure further provides methods of detecting the presence of a gene sequence expressed in a gut and methods of treating a patient based on the same. Aspects of the present disclosure further relate to a sequencing library produced by the methods of the present disclosure.

### REFERENCE TO RELATED APPLICATIONS

This application claims the benefit of U.S. Provisional Patent Application No. 63/714,222, filed Oct. 31, 2024, herein incorporated by reference in its entirety.

### STATEMENT REGARDING FEDERALLY SPONSORED RESEARCH OR DEVELOPMENT

This invention was made with government support under grant NIH HD112396-01 awarded by the National Institutes of Health. The government has certain rights in the invention.

### FIELD OF THE INVENTION

This present disclosure relates to the field of producing a DNA sequencing library for the sequencing and analysis of mRNA in a stool sample, and more specifically to methods of detecting the presence of a human gene sequence expressed in a gut from a stool sample and treating a patient based on the same.

### BACKGROUND OF THE INVENTION

Gastrointestinal (GI) maturation involves a continuous cascade of growth, differentiation, and renewal of epithelial cells. The ability to accurately elucidate changes in host intestinal mucosal and resident immune cells in response to varying conditions is important in many contexts, including the growth and development of infants. The lack of robust non-invasive approaches to repeatedly access tissue along the intestinal tract has hampered the study of normal gut development as well as responses in the gut to dietary or medical interventions.

Although some non-invasive techniques to examine the host gene expression profile of the GI mucosa have been explored, there remains a significant need in the art for a comprehensive, flexible, and reliable approach to generate host gene expression profiles derived from stool samples at a sequencing depth needed to inform clinical decisions. Accurately generating host gene expression profiles from stool samples requires effectively stabilizing the sample, selecting only gene sequences expressed from the host, ensuring robust gene yield, and accurately mapping and analyzing the resulting data. Limitations in any one of these steps can significantly impair the quality of results or completely compromise the usefulness thereof. There are currently no methods known in the art capable of defining the spectrum of intestinal phenotypes to inform directed interventions based on host gene expression obtained from a stool sample.

### SUMMARY OF THE INVENTION

In one aspect the present disclosure provides, a method of producing a sequencing library from human mRNA in a stool sample, the method comprising: treating a human stool sample with a stool stabilizing reagent; isolating RNA from the human stool sample treated with the stool stabilizing reagent based on the presence of a polyadenylated (polyA) tail; obtaining a sample of isolated RNA comprising less than about 200 ng of polynucleotides; and reverse transcribing the human mRNA using poly A-targeting primers and incorporating a unique molecular identifier into the reverse transcribed human mRNA. In one embodiment, the steps of obtaining a sample of isolated RNA comprising less than about 200 ng of polynucleotides, and reverse transcribing the human mRNA using polyA-targeting primers and incorporating a unique molecular identifier into the reverse transcribed human mRNA, are repeated to produce a plurality of sequencing libraries. In some embodiments, such method steps are repeated about two or more times, about three or more times, about four or more times, about five or more times, or about six or more times. In a further embodiment, the number of human genes detectable by the plurality of sequencing libraries relative to a single sequencing library is at least about 2-fold greater. In another embodiment, the stool stabilization reagent inhibits degradation of polynucleotides. In specific embodiments, inhibiting degradation of polynucleotides comprises inhibiting DNase and RNase activity. In yet another embodiment, the sample of the isolated RNA comprises less than about 175 ng, less than about 150 ng, or less than about 125 ng of polynucleotides. In other embodiments, the sample of the isolated RNA comprises about 100 ng of polynucleotides. In further embodiments, provided herein is a method of detecting the presence of a human gene sequence expressed in a human gut, the method comprising: sequencing the plurality of sequencing libraries; and mapping the sequences produced using a computer algorithm. In certain embodiments, the computer algorithm comprises an adjusted mismatch penalty or an adjusted match bonus setting. In specific embodiments, the mapping results in an alignment rate of at least about 10%, at least about 20%, at least about 30%, at least about 40%, at least about 50%, or at least about 60%.

A sequencing library produced by the methods disclosed herein is also provided, for example, wherein the library wherein the library comprises polynucleotide sequences complementary to at least about 3,000 human protein coding genes, at least about 4,000 human genes, at least about 5,000 human genes, or at least about 6,000 human genes. In some embodiments, the human stool sample is from an infant, such as a preterm infant; or the human stool sample is collected following a dietary intervention, e.g. administering human milk, infant formula, modified infant formula. In further embodiments, the modified infant formula comprises bioactive proteins, bioactive fats, bioactive carbohydrates, prebiotics, fermentable substrates, human milk oligosaccharides, probiotics, live microbes, fecal microbial transplants, or a combination of any thereof. In other embodiments, the human stool sample is collected following a medical intervention, such as a cesarean delivery, antibiotic administration, or intestinal surgery or resection, including for necrotizing enterocolitis.

In another aspect, a method of sequencing a nutrient absorption gene from an infant stool sample, the method comprising: treating an infant stool sample with a stool stabilizing reagent; isolating RNA from the infant stool sample treated with the stool stabilizing reagent based on the presence of a polyadenylated (polyA) tail; obtaining a sample of isolated RNA comprising less than about 200 ng of polynucleotides; reverse transcribing the human mRNA using polyA-targeting primers and incorporating a unique molecular identifier into the reverse transcribed human mRNA; repeating said steps to produce a plurality of sequencing libraries; and sequencing the plurality of sequencing libraries. In some embodiments, the method further comprises mapping the sequences produced using a computer algorithm; and detecting the presence of a decreased or increased level of a nutrient absorption gene mRNA relative to a control. In specific embodiments, the method further comprises detecting the presence of an increased level of SLC1A1, SLC38A1, SLC38A2, ABCG5, SLC26A2, LPL, SAR1B, SLC44A1, or BTD mRNA relative to an appropriate control. In another embodiment, the method further comprising mapping the sequences produced using a computer algorithm; and detecting the presence of a decreased or increased level of a nutrient absorption gene, a nutrient transporter gene, barrier function gene, a hypoxia-related gene, a GI ischemia-related gene, a heat shock protein gene, an HDAC response gene, a butyrate metabolism gene, an energy utilization gene, a stemness gene, or an immune response gene mRNA relative to a control.

In yet another aspect provided herein is a method of treating an infant with feeding intolerance comprising: obtaining a sample from the infant that comprises at least a first mRNA associated with feeding intolerance; detecting in the sample the presence of an increased or decreased level of the mRNA relative to a control infant that lacks feeding intolerance; and treating the infant with feeding intolerance based on the increased or decreased level of the mRNA relative to the control. In some embodiments, treating the infant comprises no treatment. In other embodiments, treating the infant comprises administering intravenous nutrition. In still other embodiments, treating the infant comprises administering a partially hydrolyzed formula or human milk treated with enzymes.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the enhanced performance of Zymo DNA/RNA Shield when compared with other RNA stabilization reagents in regard to number of genes ultimately detected in feces. Zymo Shield outperforms the most commonly used RNA Later by about 8-fold in gene output.

FIG. 2 shows the improved performance of using a polyA tail-based sequencing library kit in the detection of human gene reads. Compared to a non-polyA tail-based kit, there is a large improvement in human gene detection.

FIG. 3 shows the improved performance in human gene detection when limiting fecal RNA input into the sequencing library kit.

FIG. 4 shows the improvement in human gene detection when using a plurality of sequencing libraries at a lower RNA input level as compared to a single library at a higher RNA input level.

FIG. 5 shows the total number of genes detected with and without the use of unique molecular identifiers, highlighting the problem of PCR duplication in low input pipelines. These UMIs remove PCR bias to significantly improve data quality.

FIG. 6 shows the improved performance in alignment rate, which corresponds to human reads detected, by using Bowtie2 with adjusted mismatch penalty and match bonus settings determined for each individual experiment, as compared to results obtained using the unadjusted algorithm.

FIG. 7 shows differential expression data related to human infants and adults (preterm; 2-week old; 5-month old; 4-year old infant; adult) produced using the methods disclosed herein. The expression of genes involved in the absorption of nutrients in the gut (using a heat map) is shown, which serve as putative biomarkers to inform treatment decisions.

FIG. 8 shows differential expression data related to human infants and adults (preterm; 2-week old; 5-month old; 4-year old infant; adult) produced using the methods disclosed herein. The expression of genes involved in the immune defense response in the gut (using a heat map) is shown, which serve as putative biomarkers to inform treatment decisions.

FIG. 9 shows differential expression data related to human infants and adults (preterm; 2-week old; 5-month old; 4-year old infant; adult) produced using the methods disclosed herein. The expression of cell-marker genes (i.e. genes that are highly expressed in only one particular type of cell) in the gut (using a heat map) is shown.

FIG. 10 shows differential expression data related to human infants and adults (preterm; 2-week old; 5-month old; 4-year old infant; adult) produced using the methods disclosed herein. The expression of genes involved in glucose homeostasis and glucose metabolism in the gut (using a heat map) is shown, which serve as putative biomarkers to inform treatment decisions.

FIG. 11 shows differential expression data related to human infants and adults (preterm; 2-week old; 5-month old; 4-year old infant; adult) produced using the methods disclosed herein. The expression of genes involved in short chain fatty acid metabolism and transport in the gut (using a heat map) is shown, which serve as putative biomarkers to inform treatment decisions.

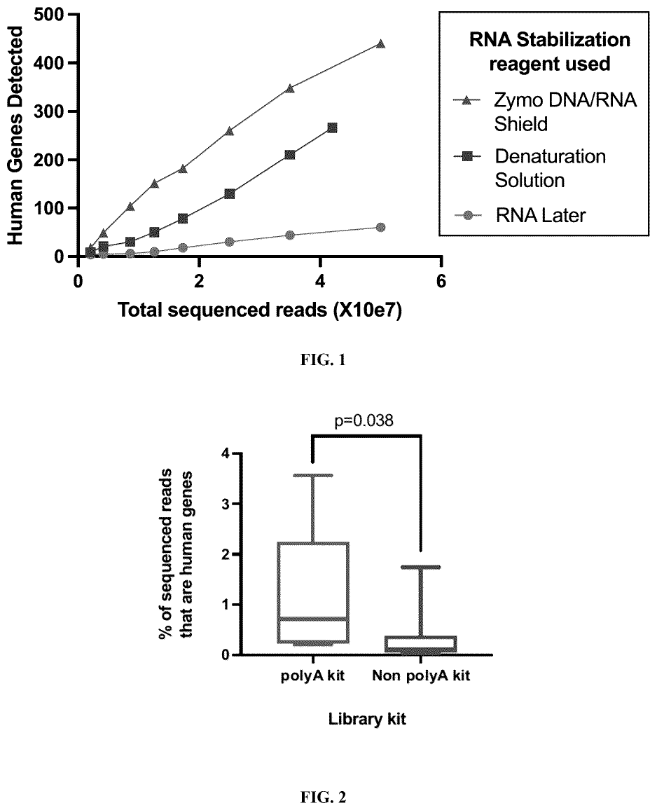

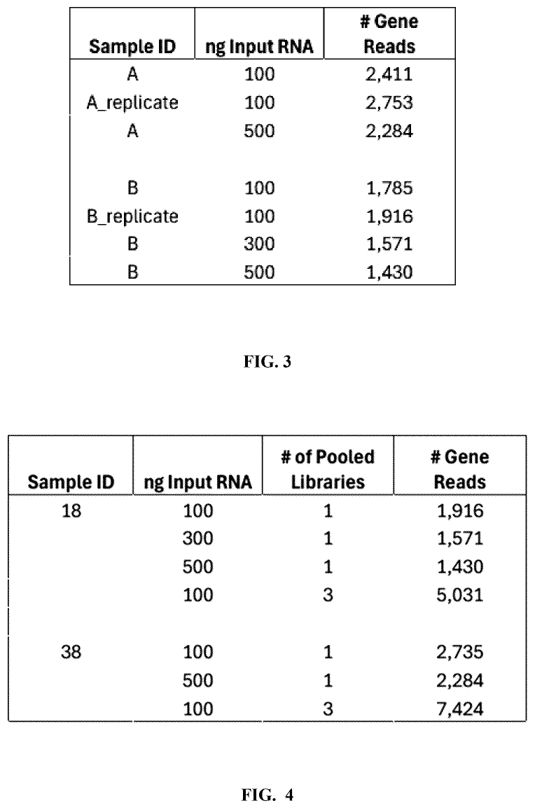

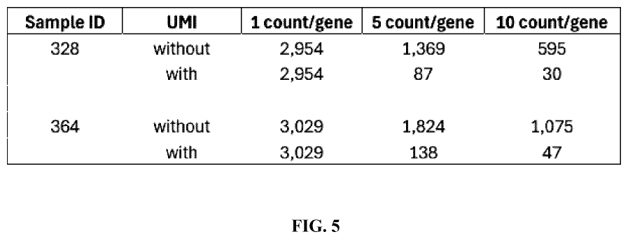

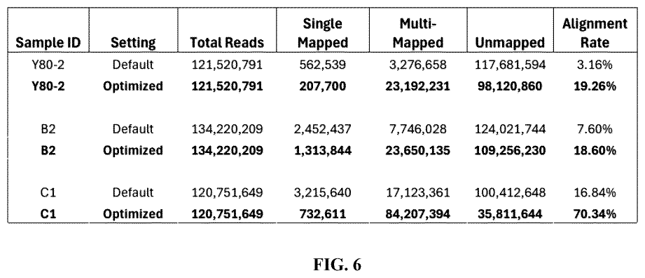

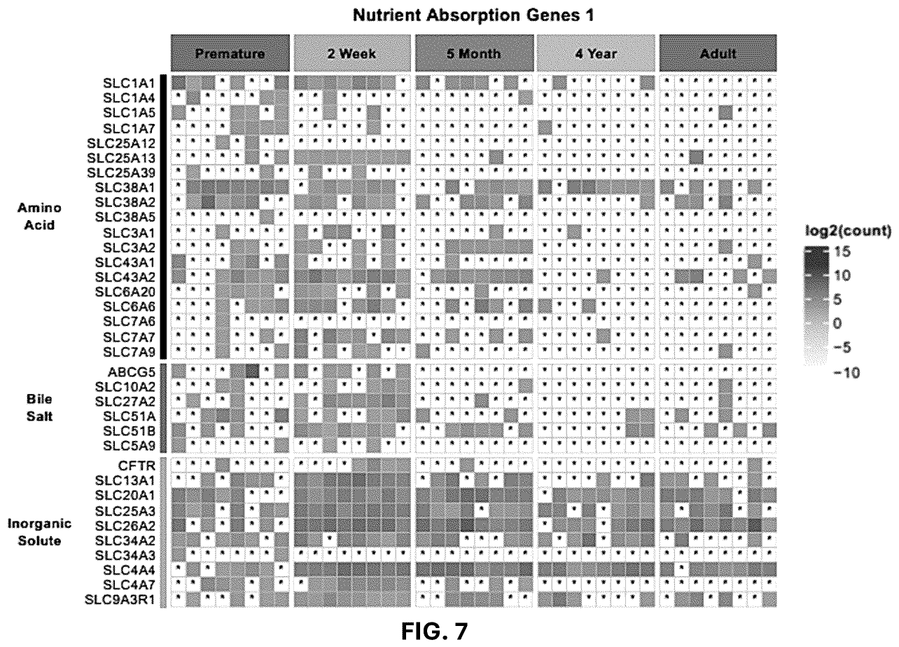

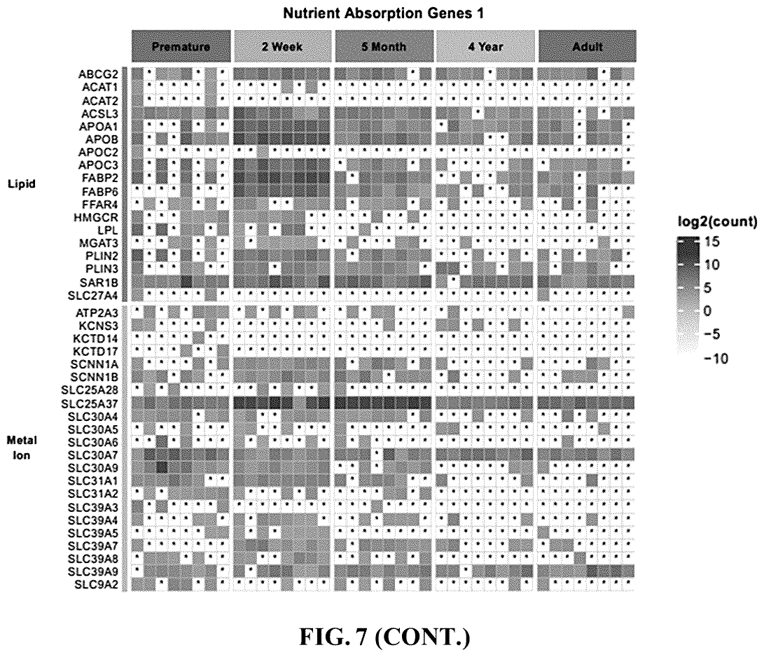

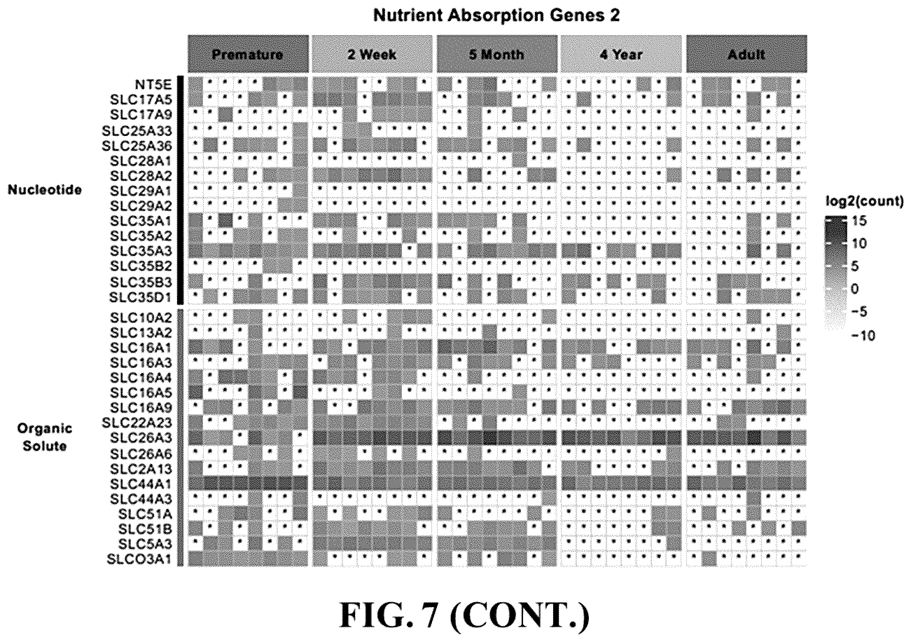

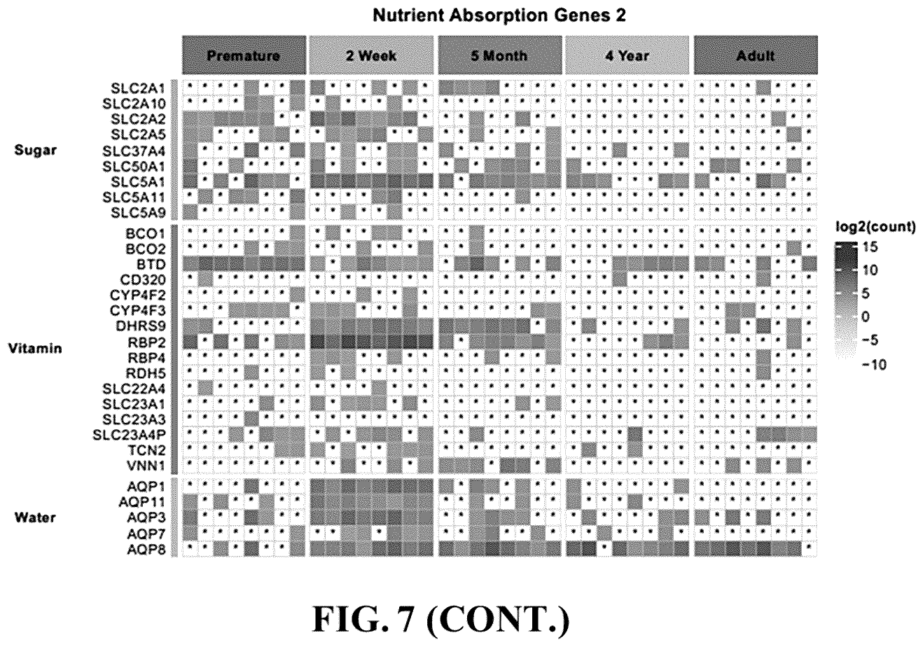

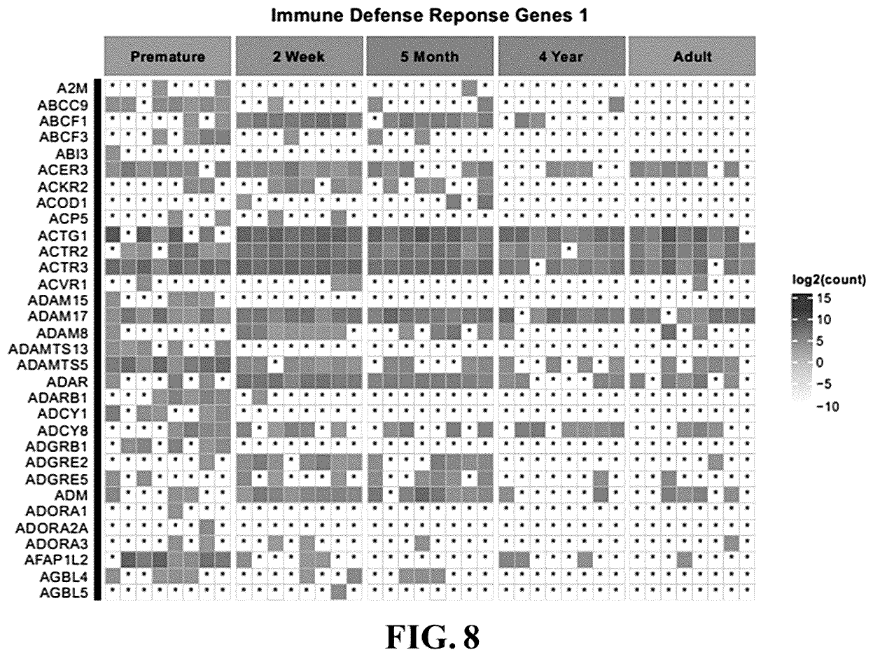

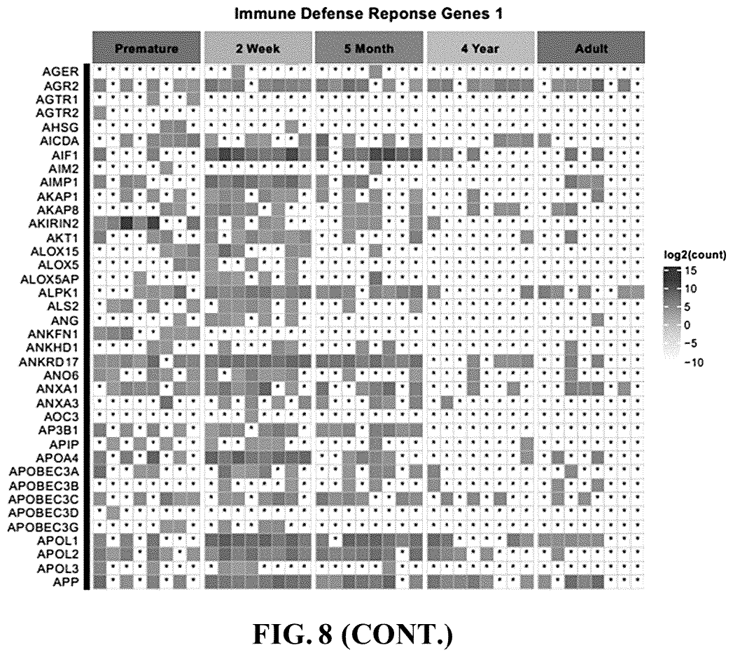

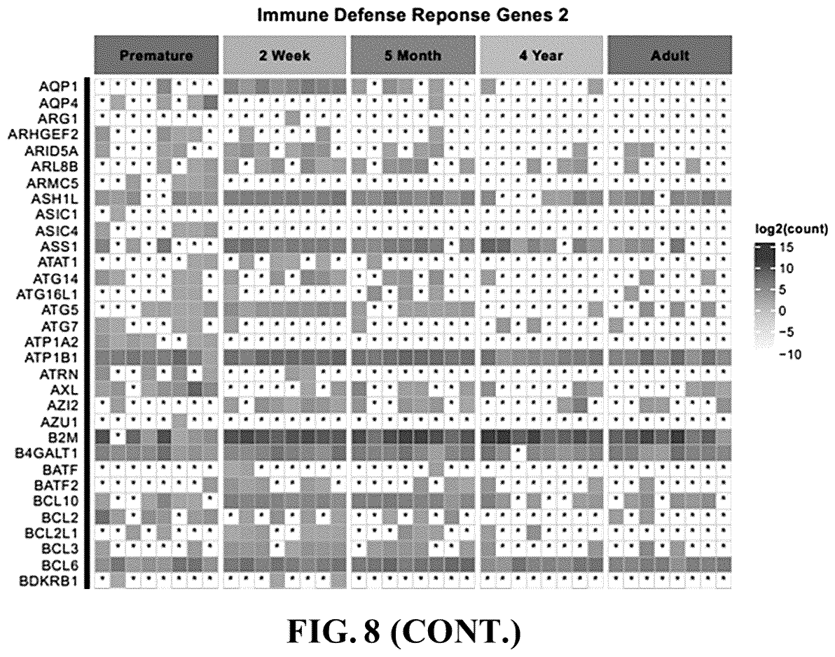

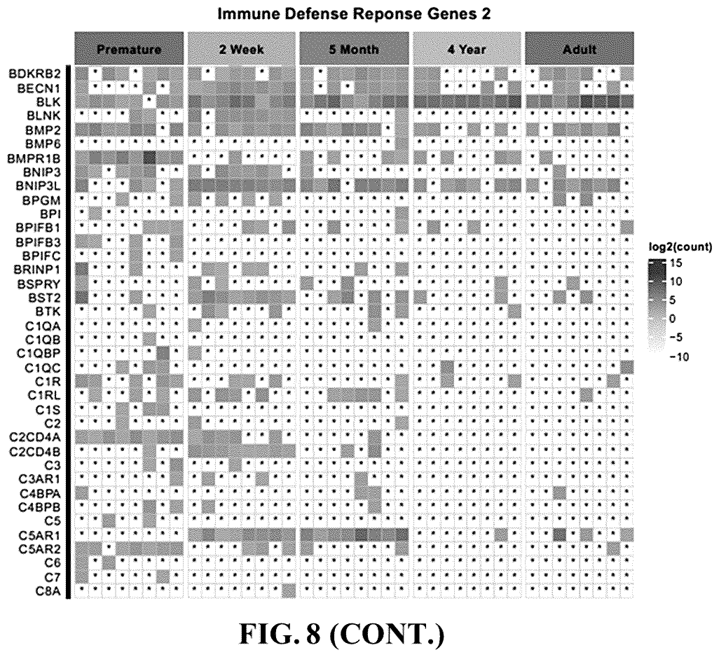

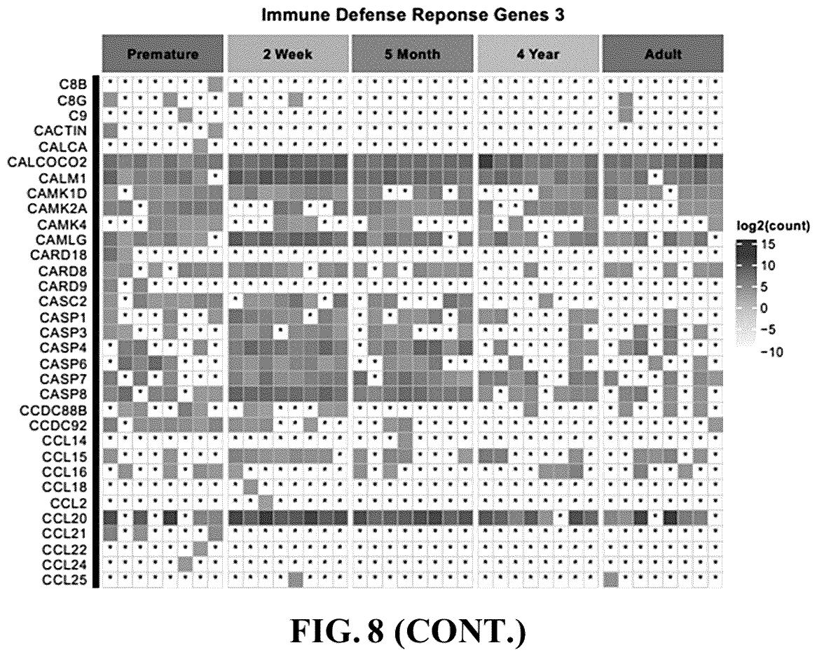

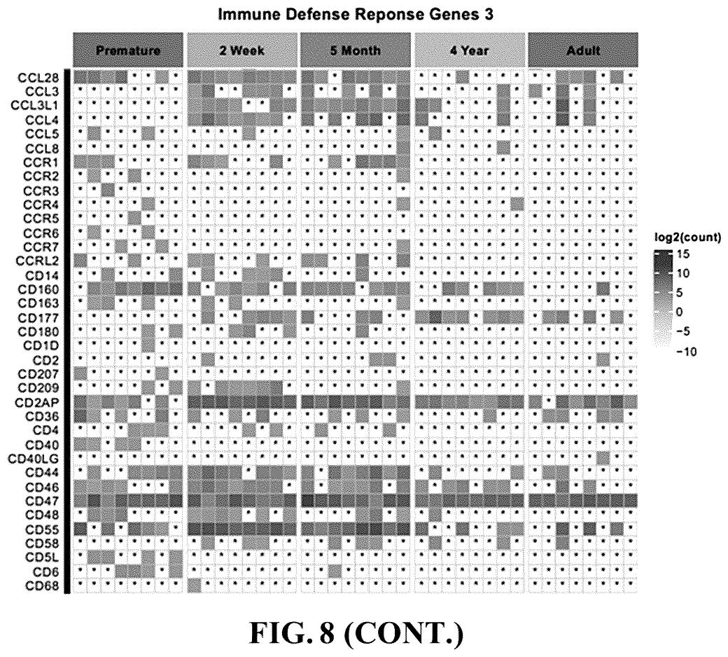

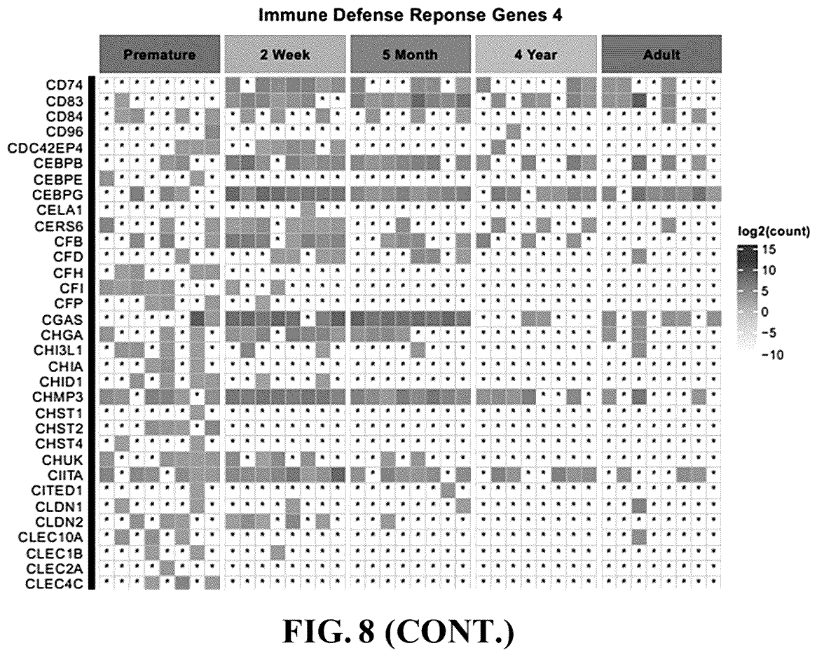

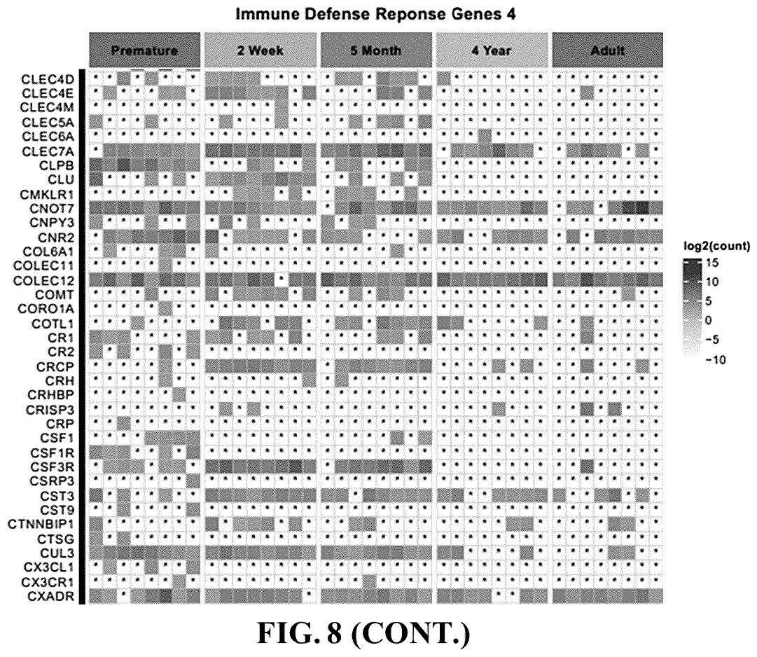

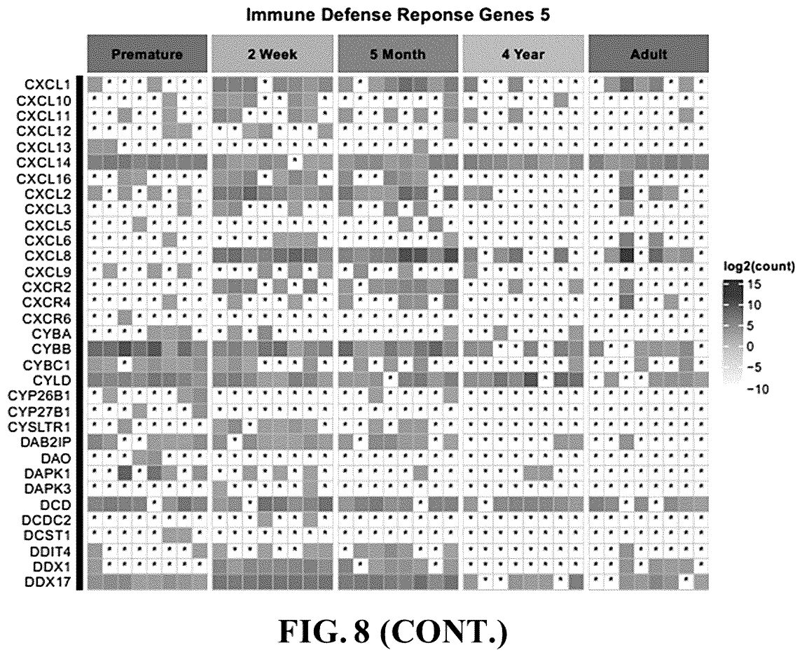

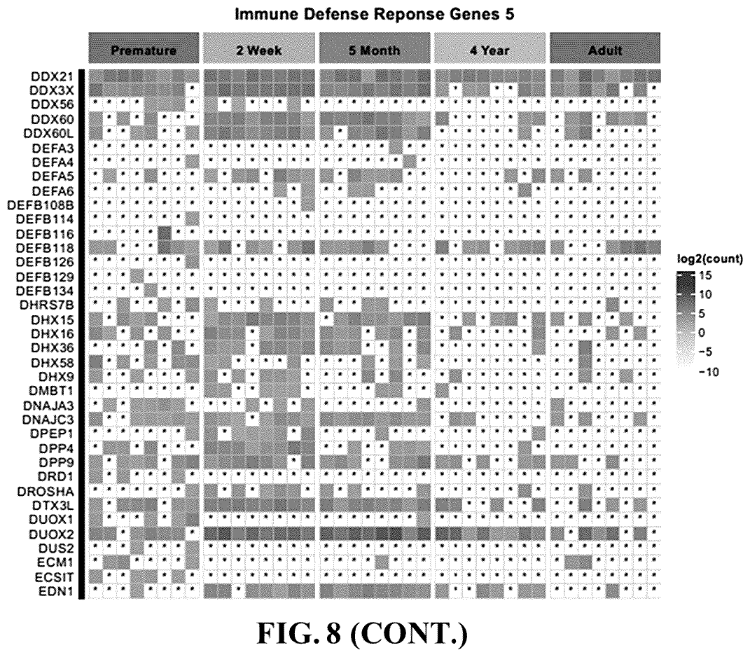

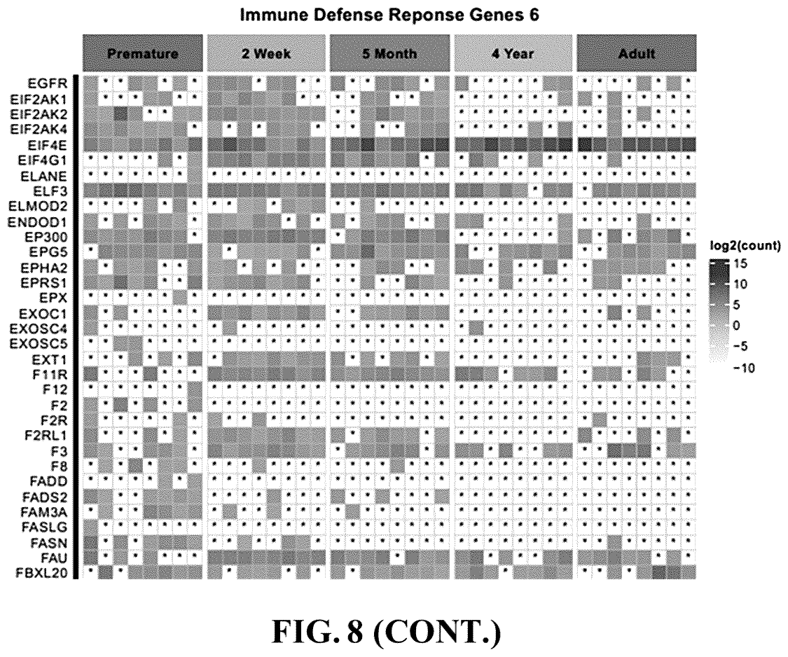

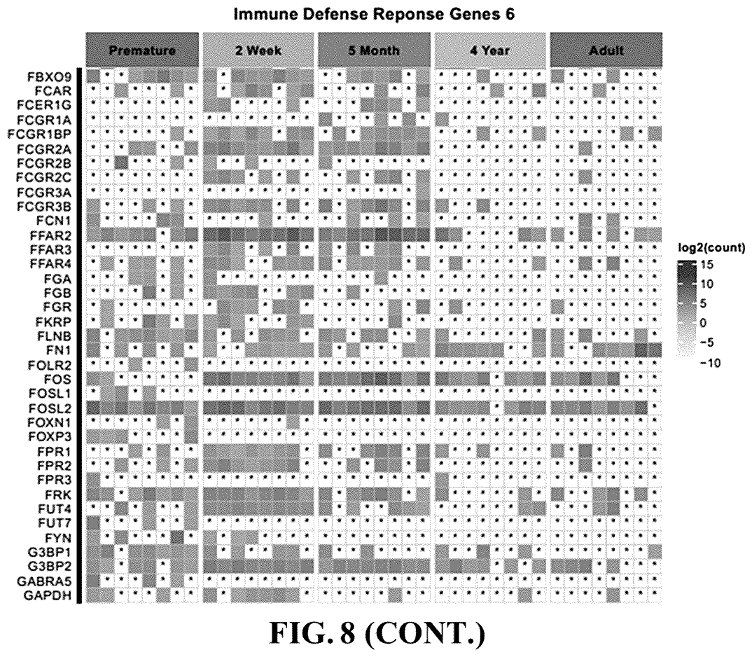

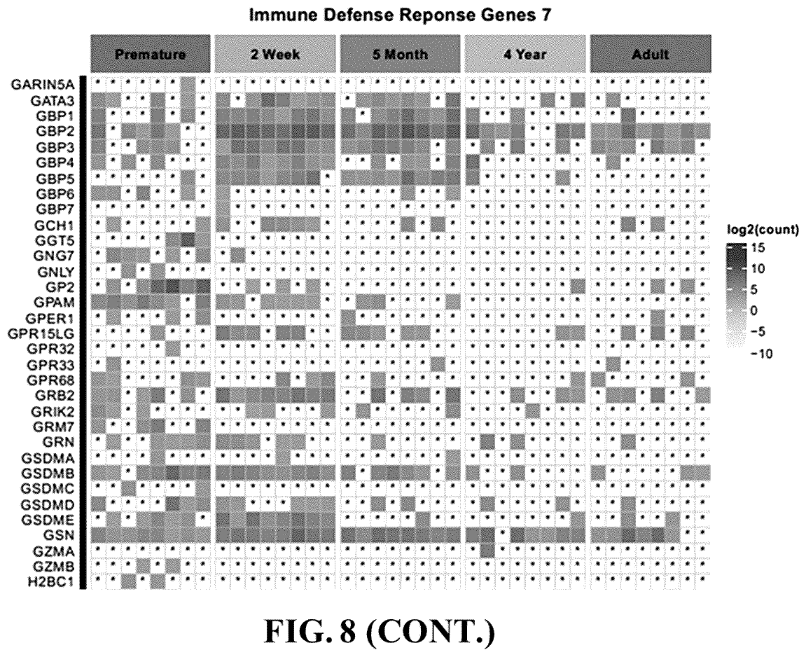

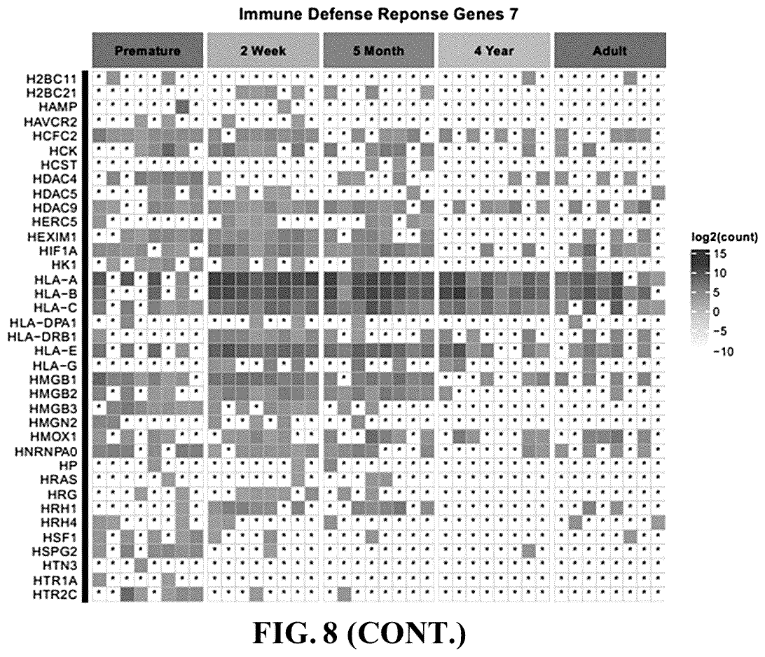

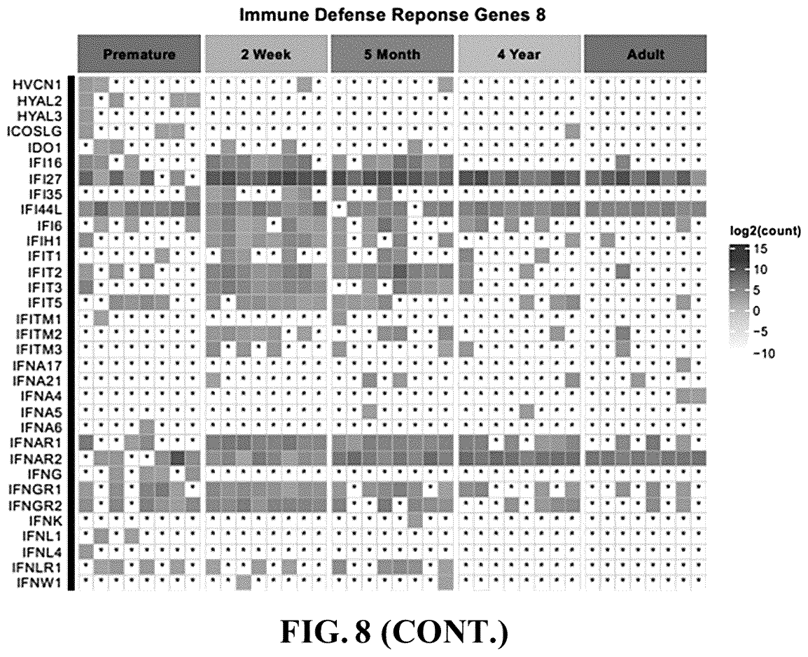

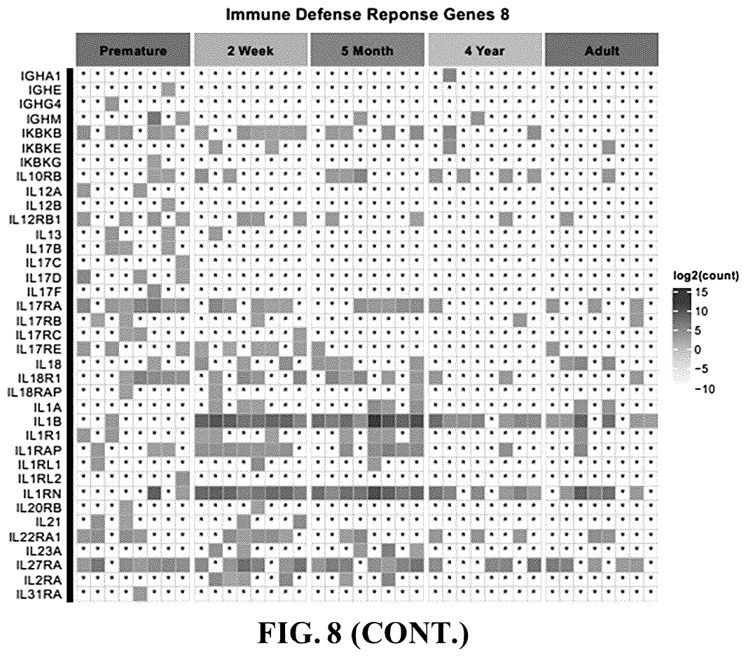

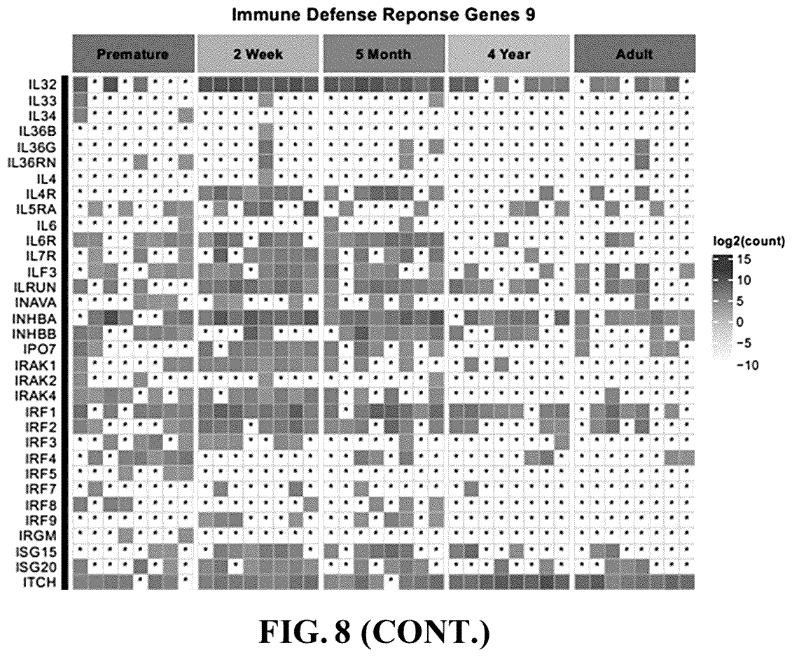

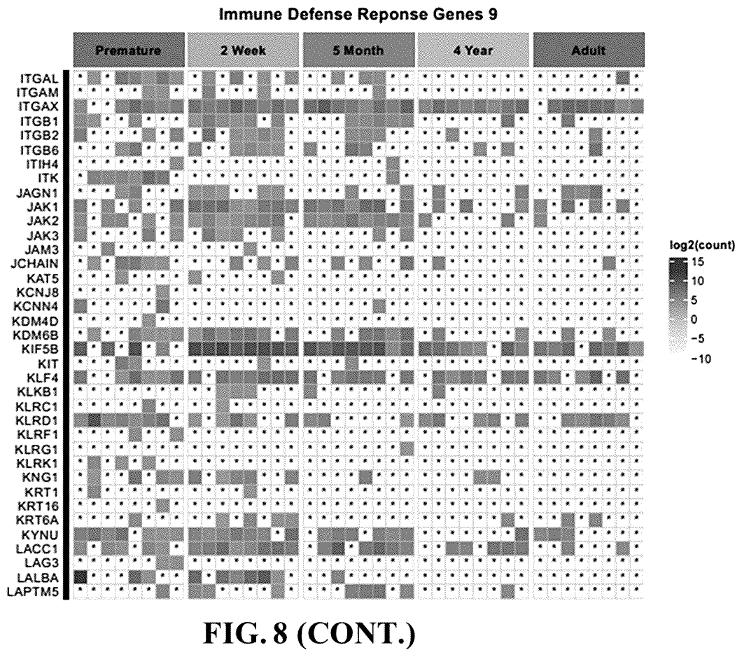

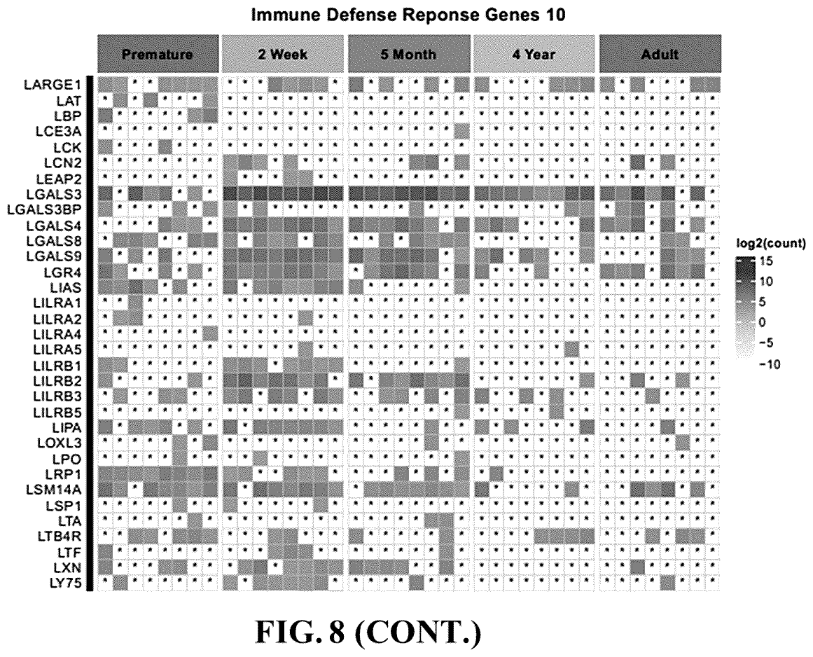

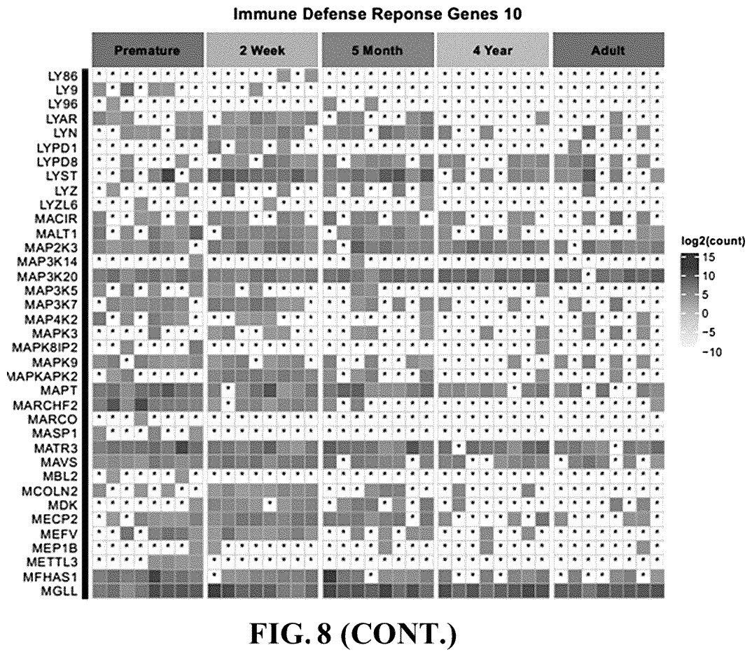

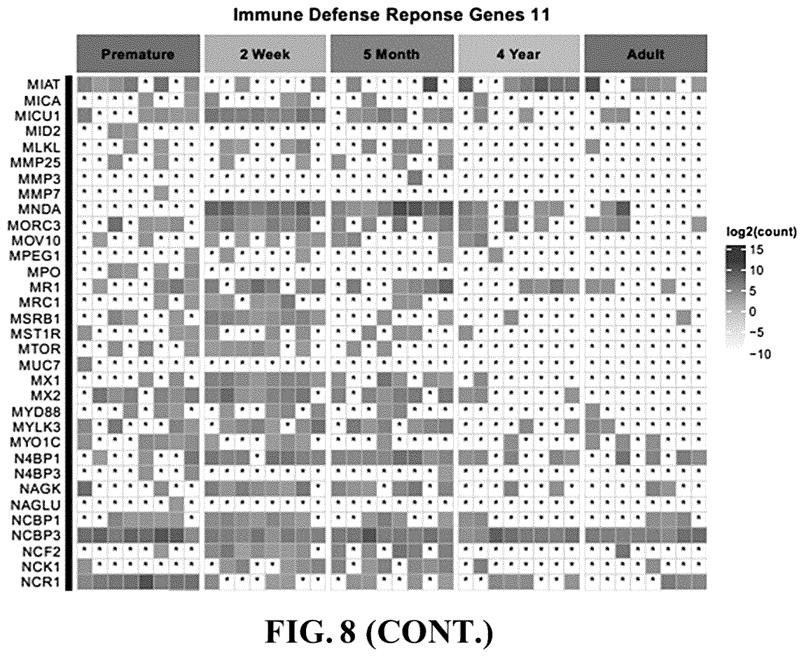

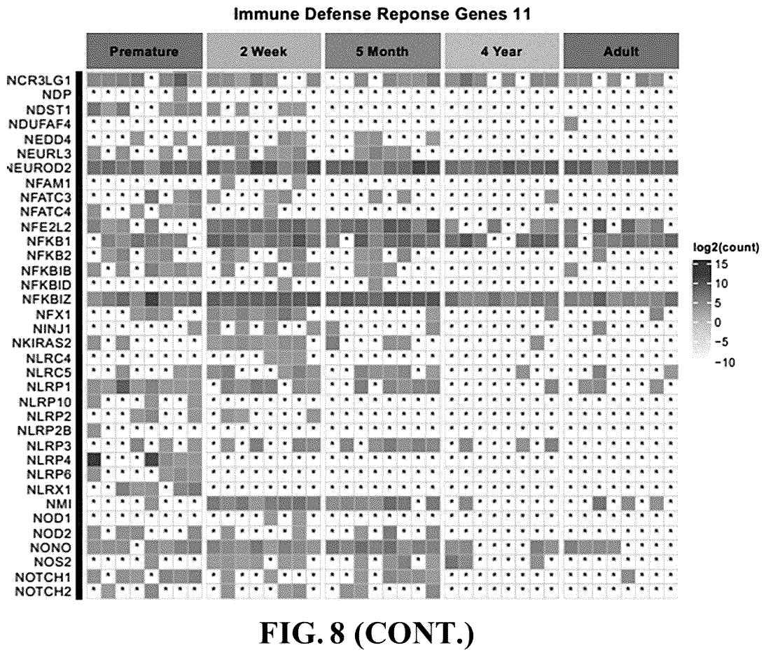

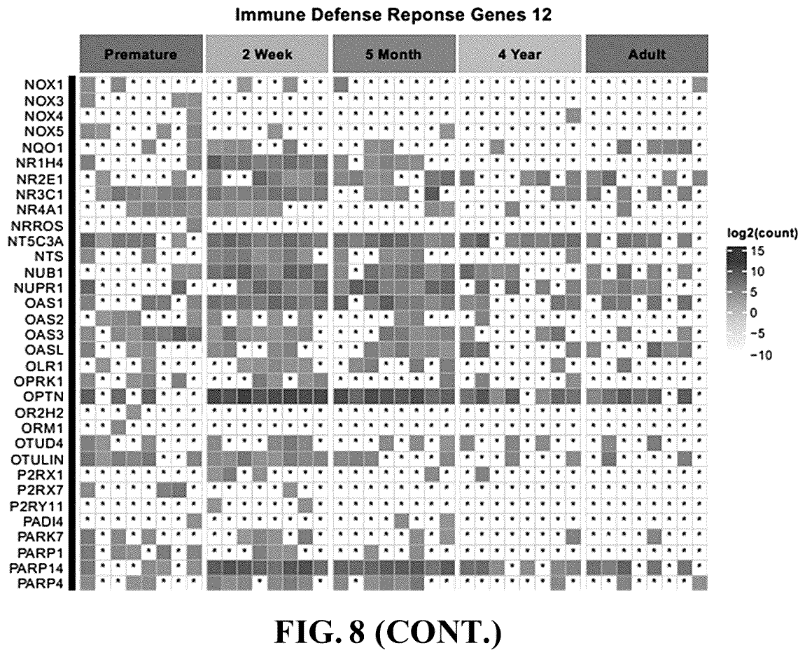

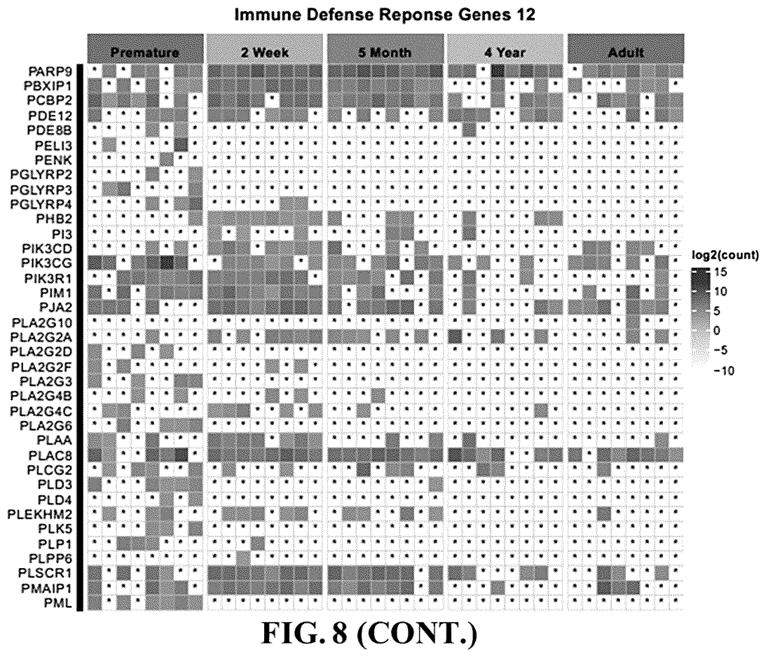

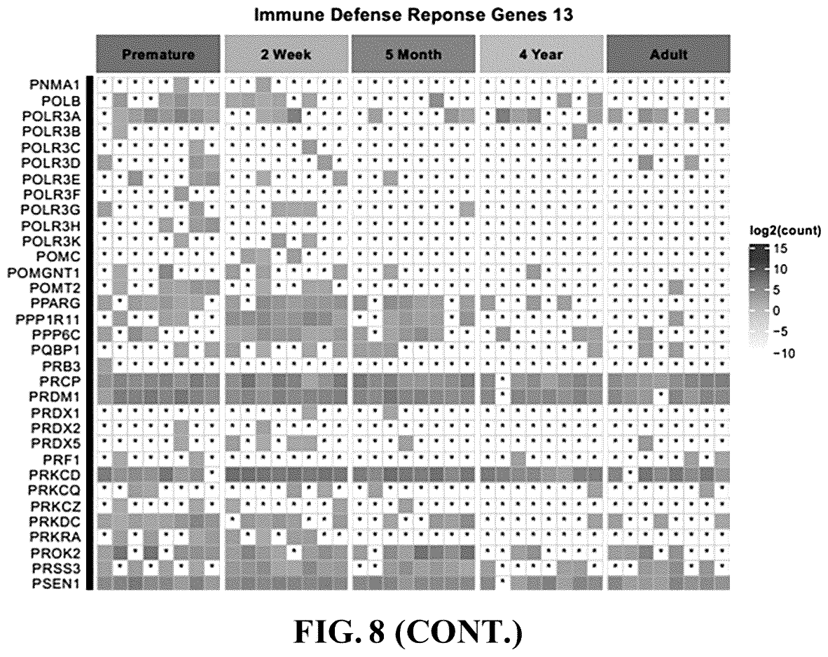

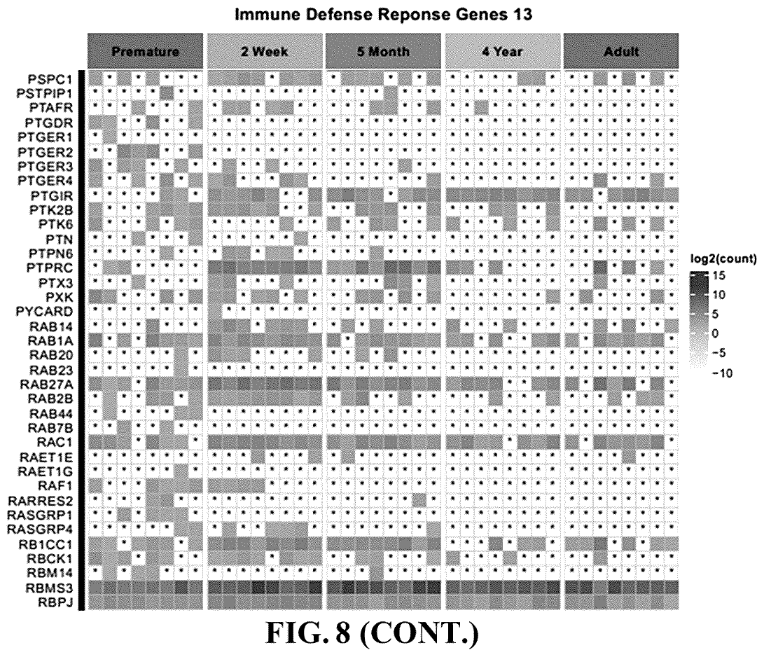

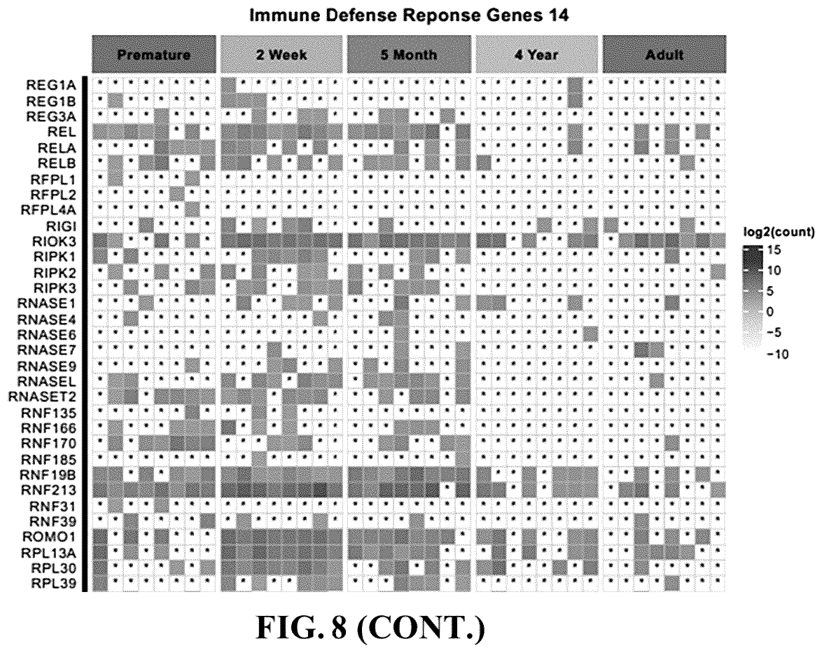

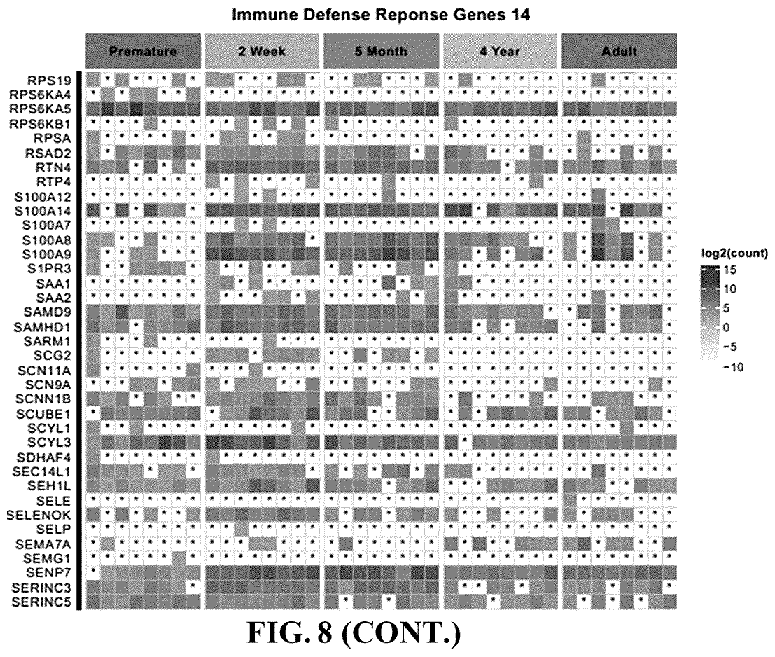

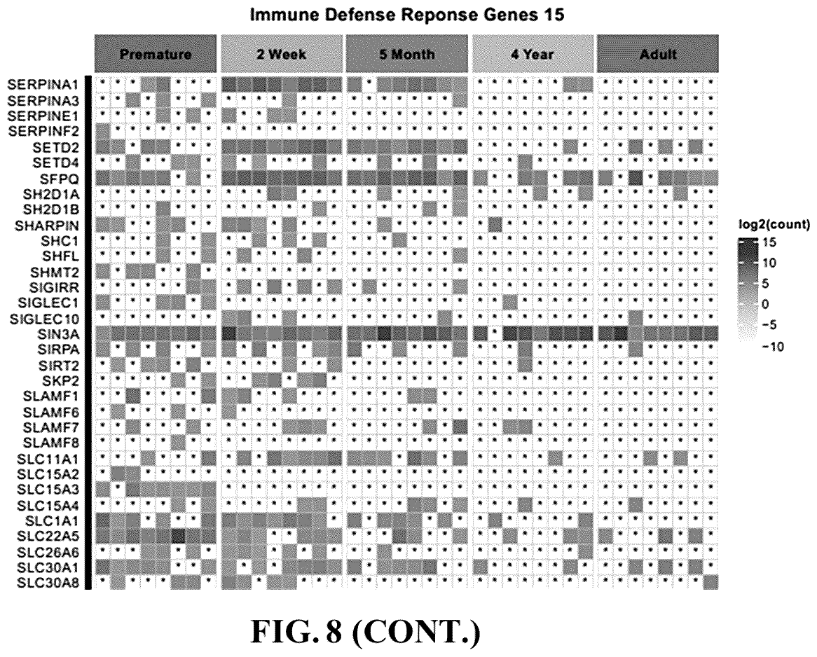

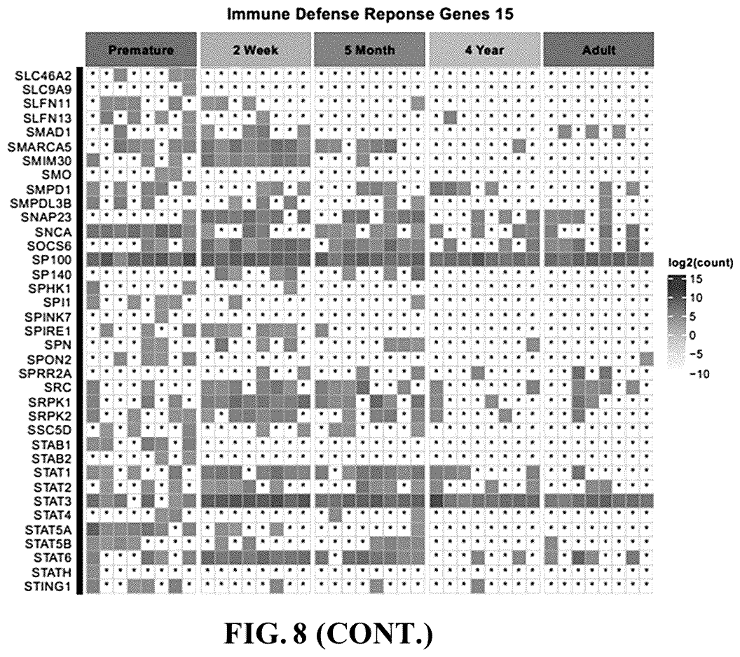

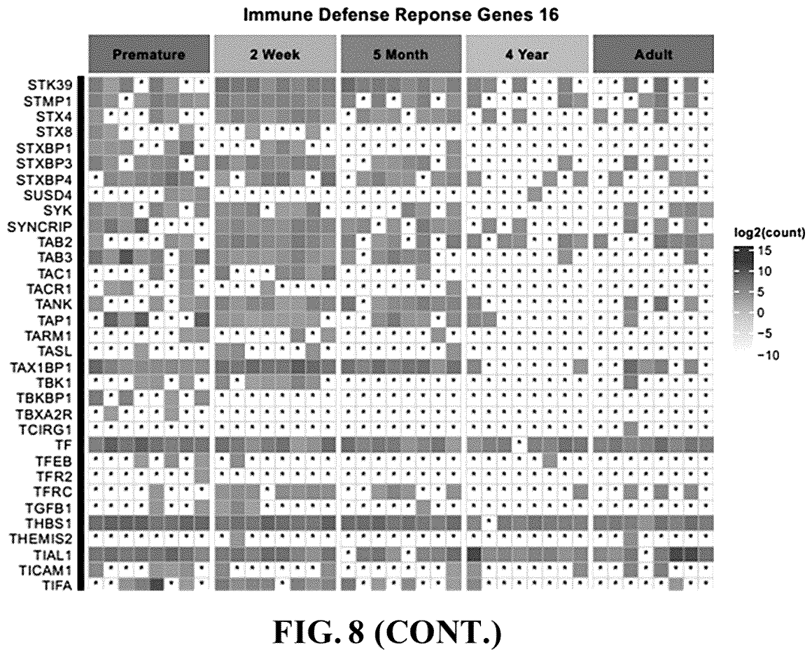

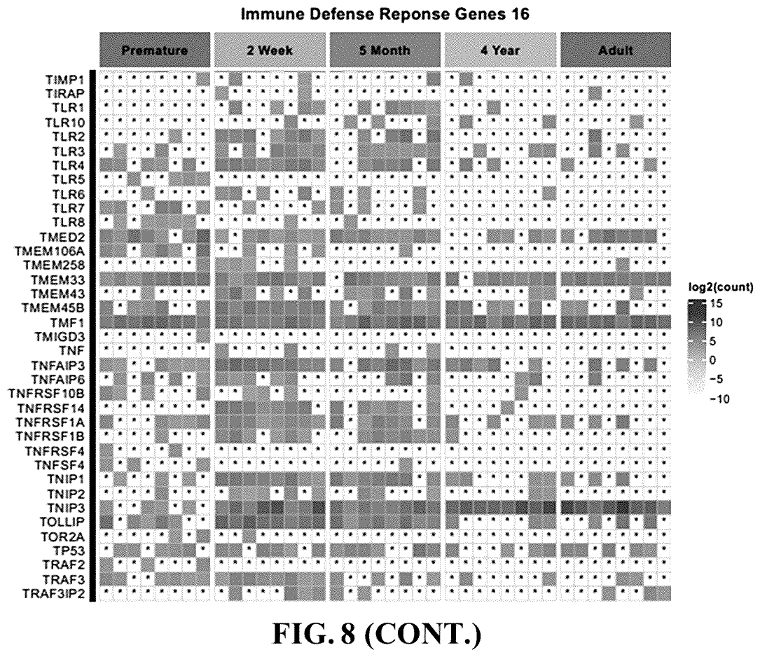

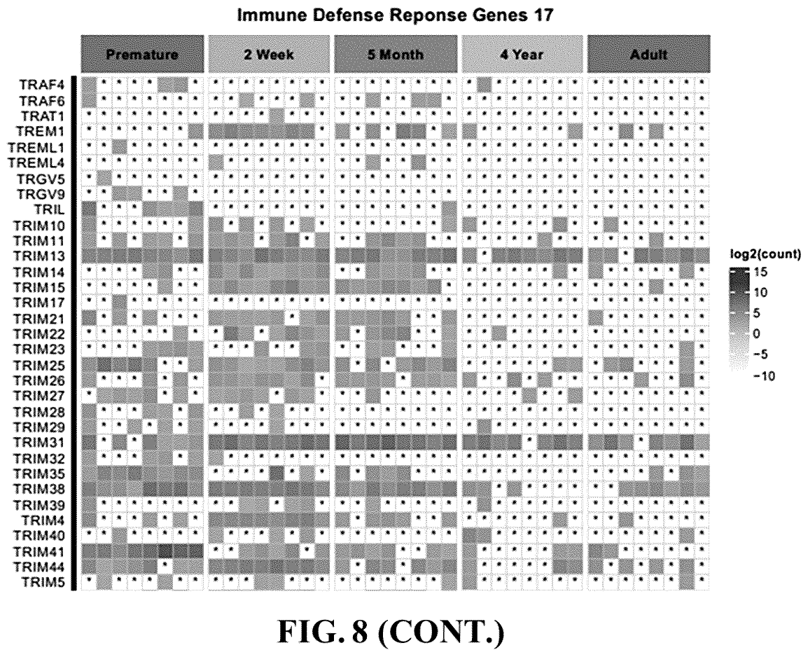

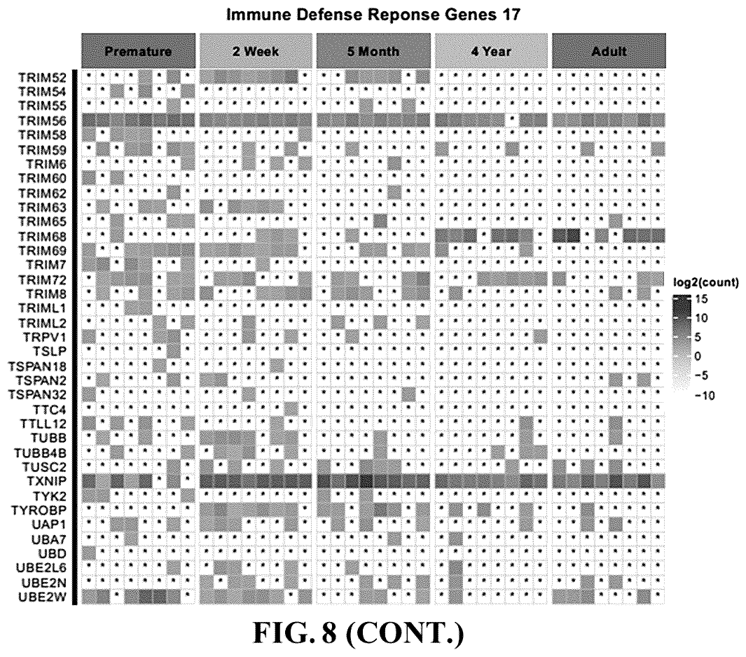

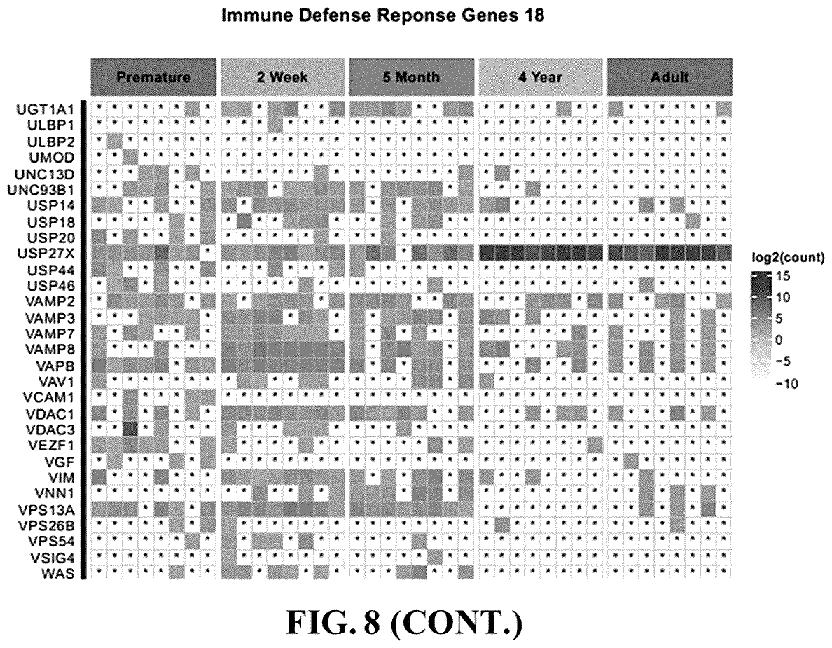

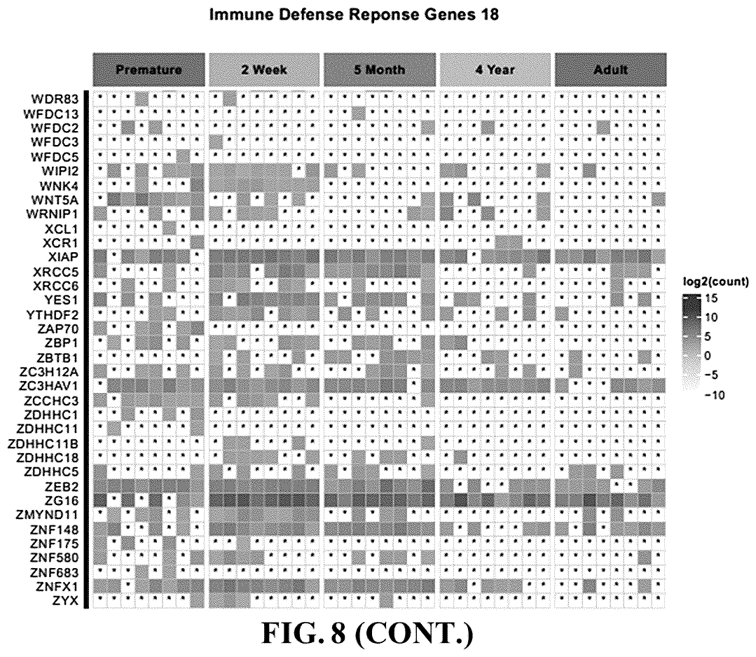

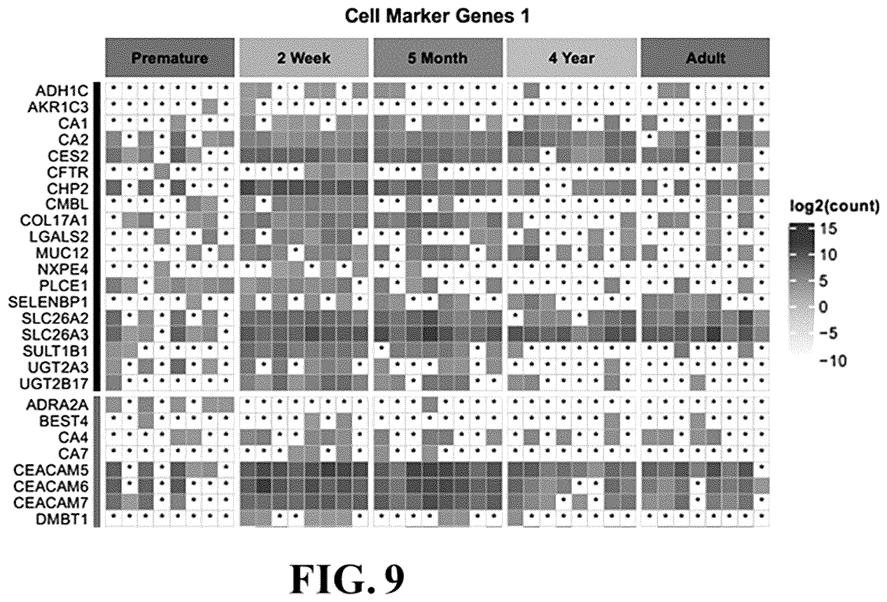

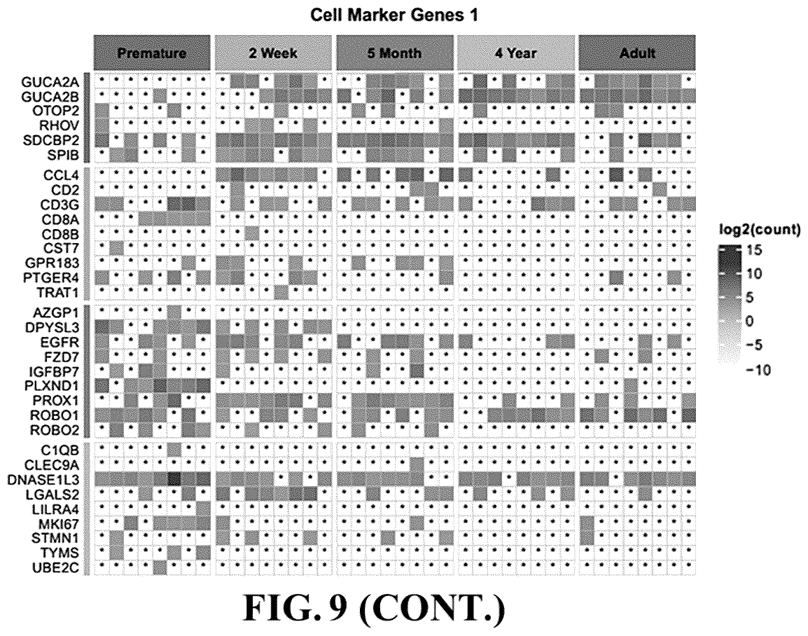

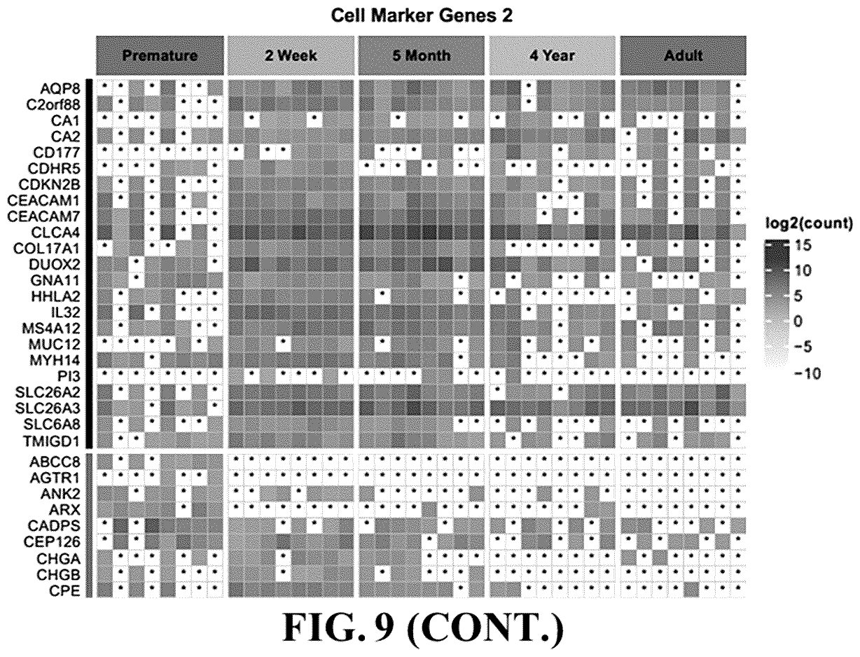

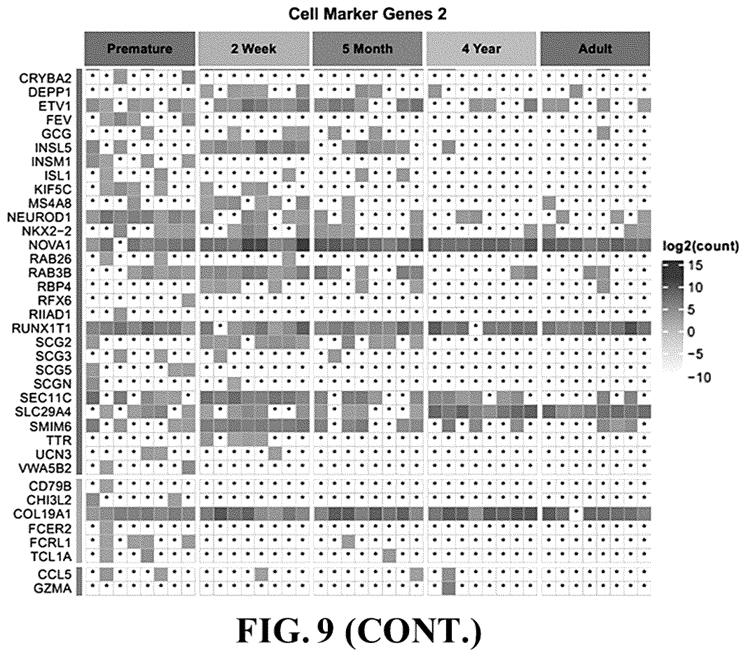

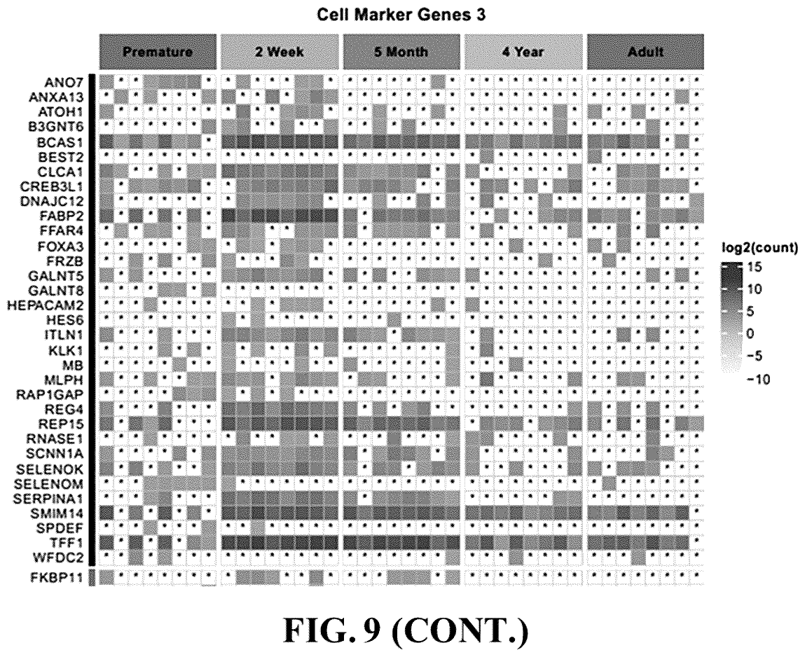

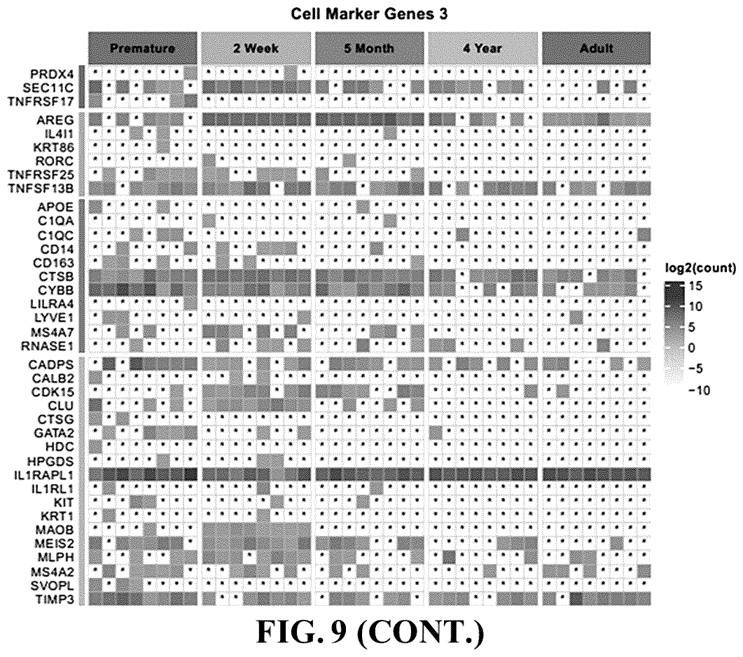

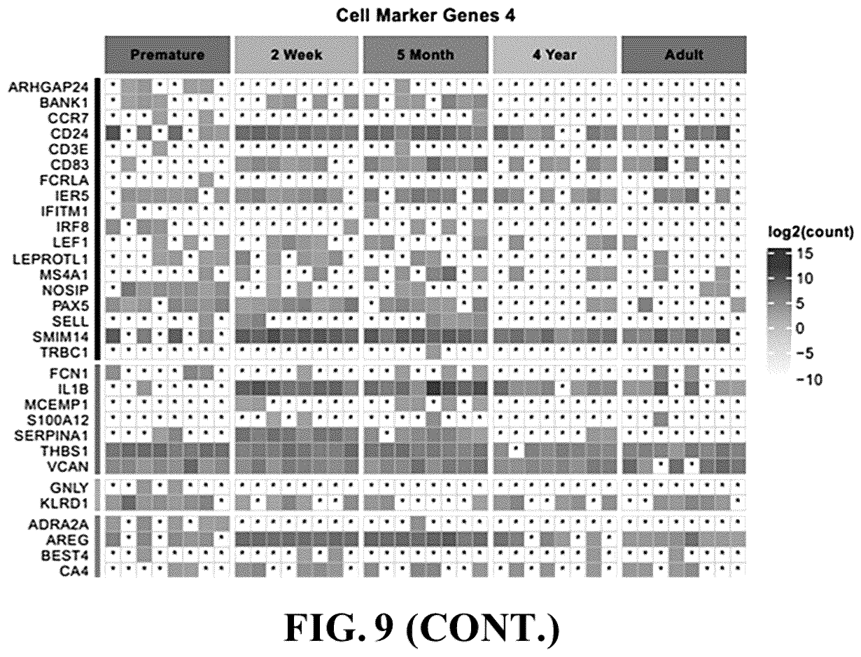

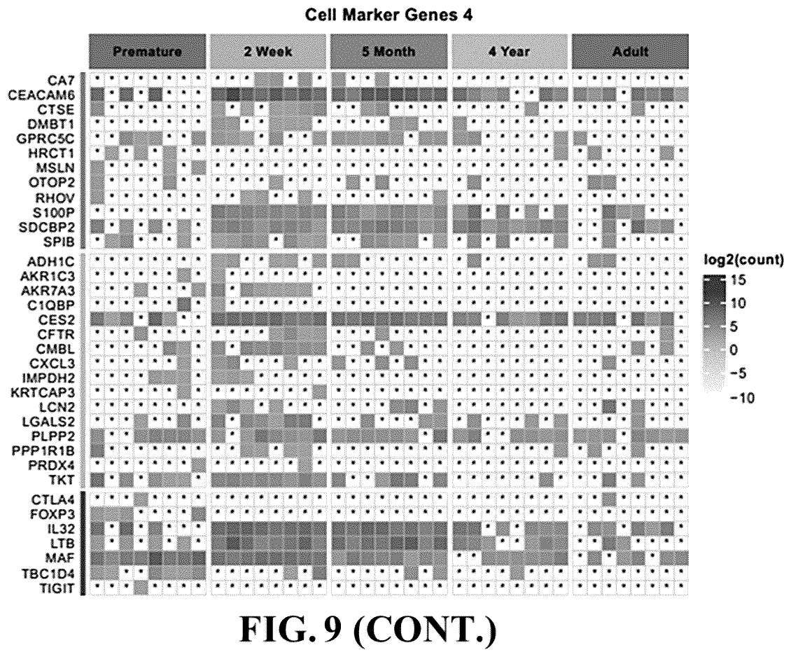

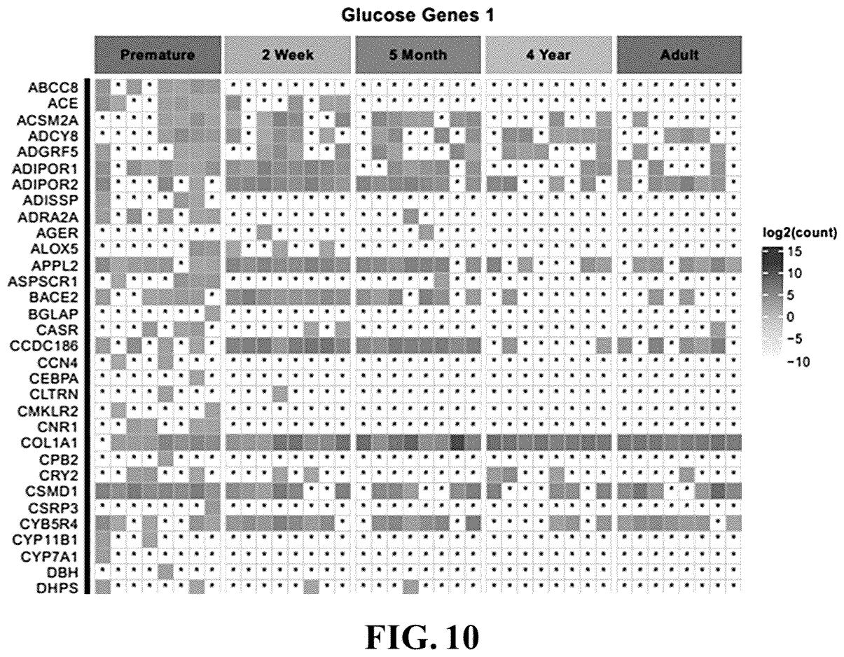

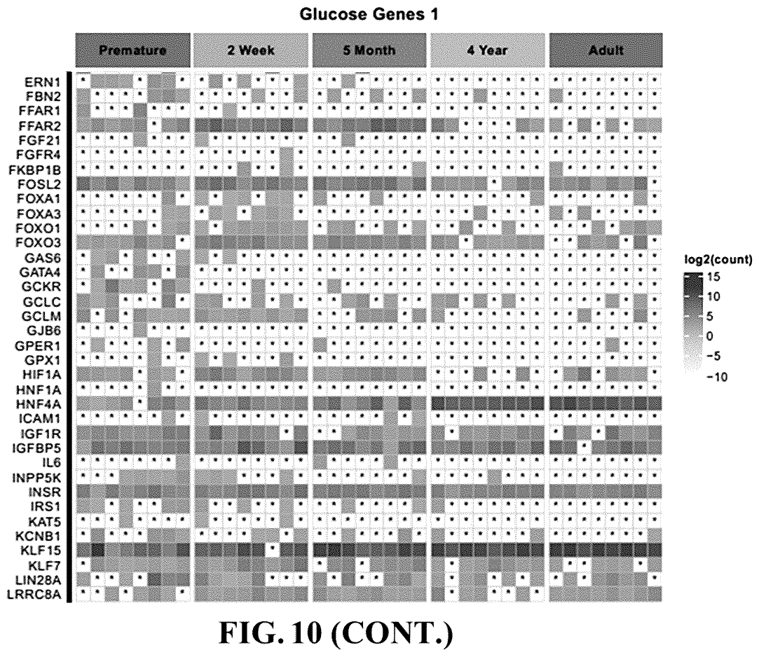

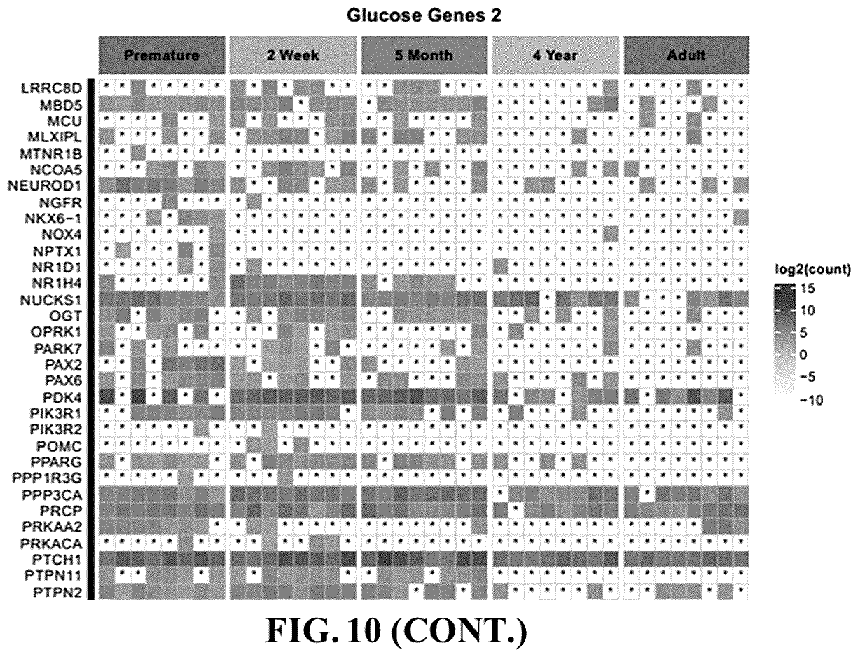

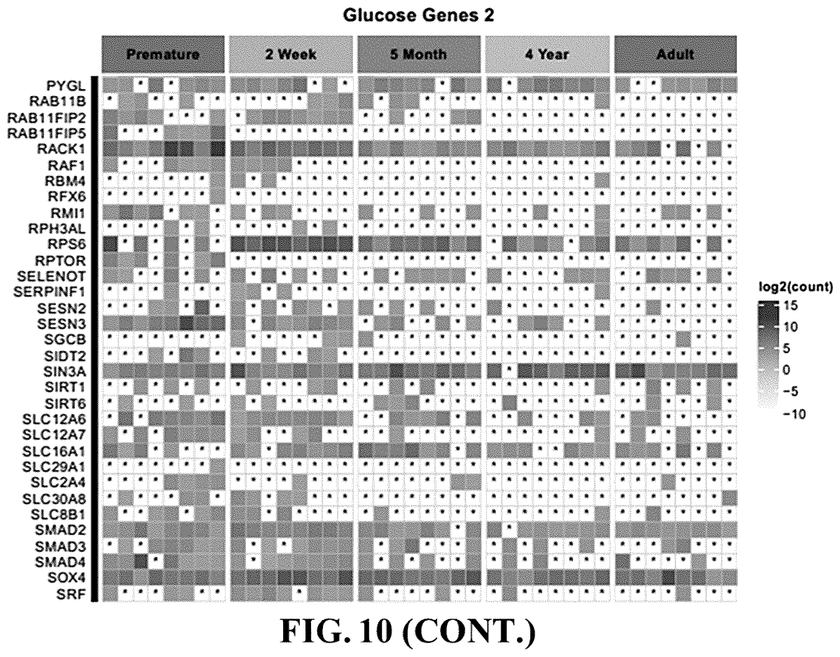

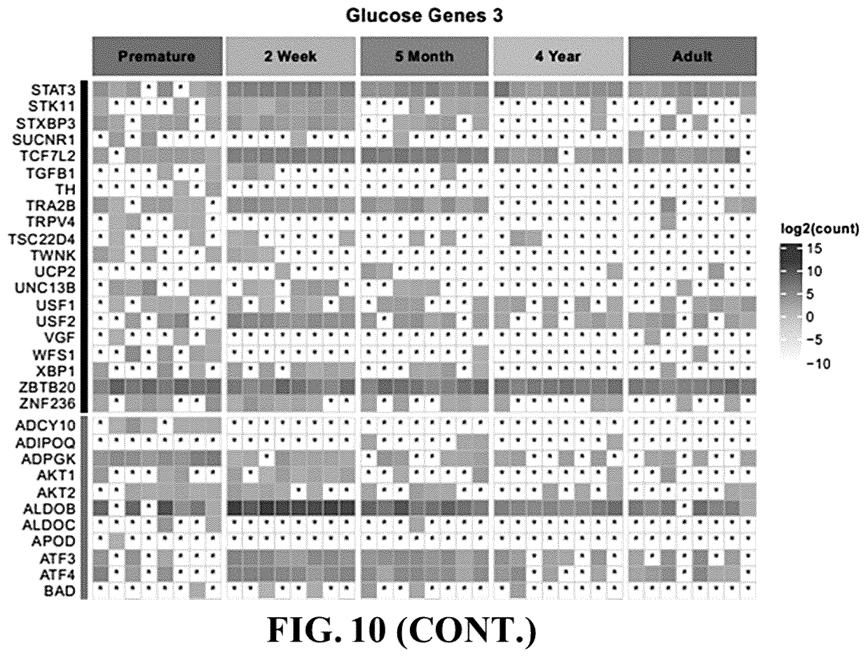

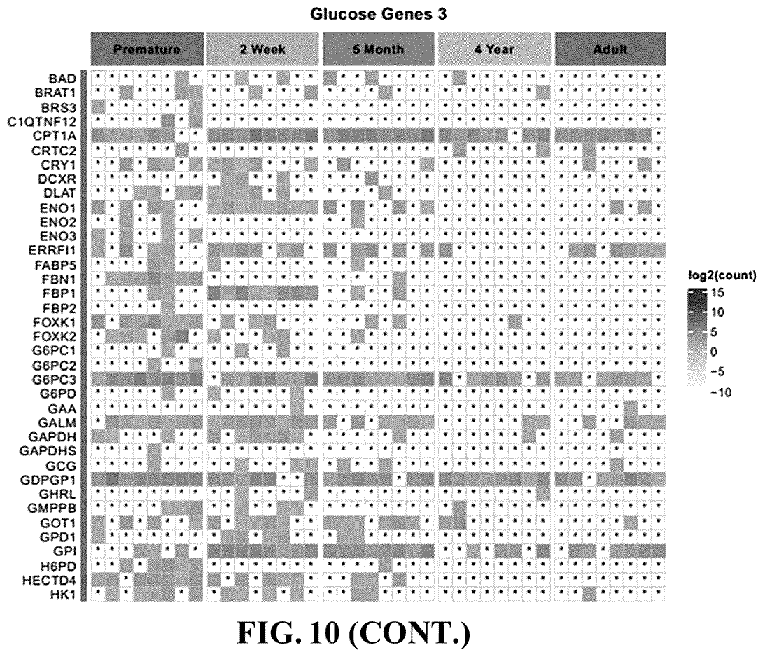

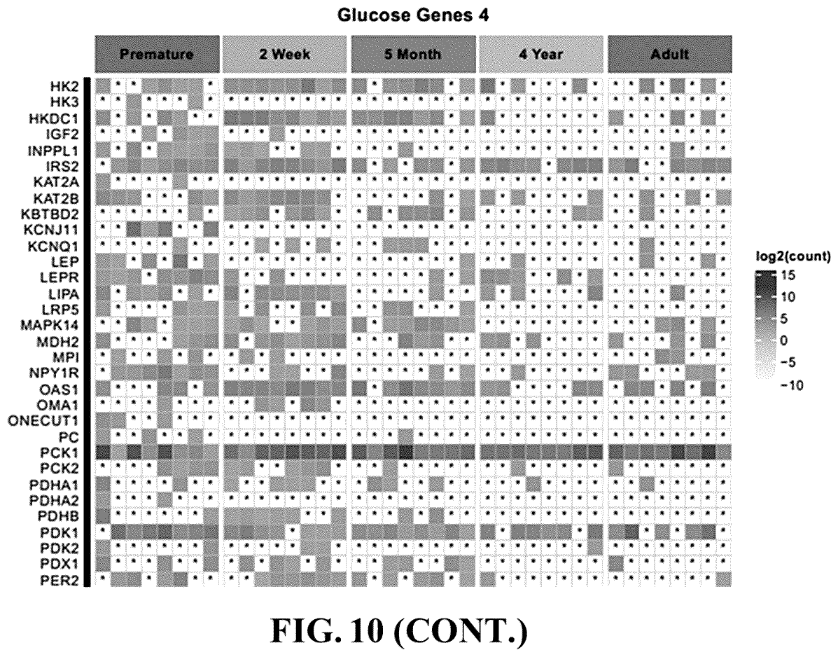

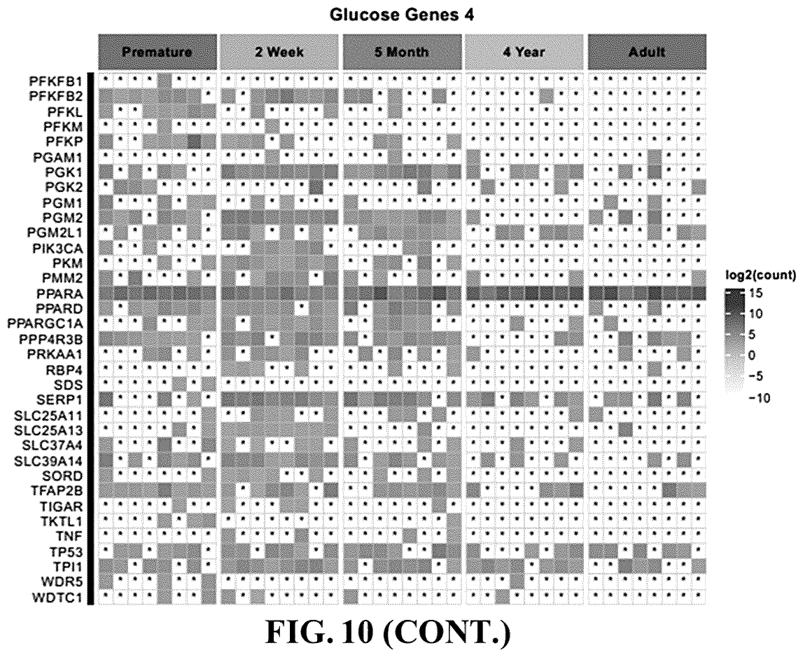

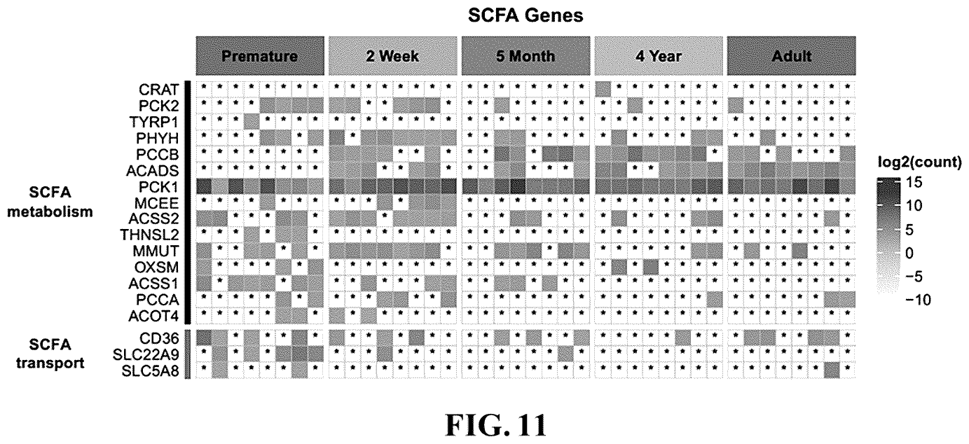

### DETAILED DESCRIPTION OF THE INVENTION

The methods described herein facilitate personalized gastrointestinal insights and dietary and medical interventions using non-invasive data collection and analysis techniques. The methods provided utilize stool-derived exfoliated cells to monitor the gut transcriptome (exfoliome) eliminating the need for invasive biopsies. The methods described herein enable gene expression analysis at a sequencing depth that surpasses any currently available methods in the art. Such methods can also provide insights into the functional interactions between diet, microbiota, and host intestinal development; and identify biomarkers and gene expression profiles that indicate the impact of development, diet, or disease on the gut environment, ultimately supporting therapy recommendations tailored to an individual's microbiome and genetic makeup. The integrative non-invasive methodologies described herein ensure that human gene expression data derived from stool samples may be accurately and repeatedly relied upon to inform treatment decisions; and allow for personalized therapeutic strategies that align with an individual's unique intestinal environment, potentially enhancing outcomes related to gut health and development.

The need for new methods to monitor gastrointestinal health is evident. Current diagnostics often rely on invasive techniques or indirect measures that are insufficient for personalized treatment. Furthermore, current methods known in the art are not able to obtain the depth and breadth of sequencing necessary to obtain actionable and reproducible insights to inform treatment decisions. In the context of infants, non-invasive techniques are even more critical, as traditional biopsies or invasive monitoring methods are not feasible. The application of the methods described herein has the potential to define the spectrum of intestinal phenotypes ranging from feeding intolerance to severe injury, such as necrotizing enterocolitis (NEC), that remains a clinical enigma and clouds definitive interpretation of research studies in this area. Such methods, in combination with the sequencing analysis workflows described herein allow for the identification of early gene biomarkers, e.g., predictors of intestinal dysfunction, to inform precision (personalized) medicine/nutrition-directed interventions.

The methods provided herein overcome the current limitations associated with host gene expression data obtained from stool samples, including the very limited numbers of human genes detected from small and large intestinal epithelial and immune cell populations, microbial contamination, PCR artifacts which compromise the quantitative assessment of gene expression and variability in sequencing library preparation. The present inventors found that the combination of method steps described herein enables the production of sequencing libraries from human mRNA in a stool sample, wherein the library comprises polynucleotide sequences complementary to significantly more human protein coding genes as compared to methods known in the art. Surprisingly, the inventors found that limiting the mRNA input in the method of producing the sequencing library increases the number of genes identified. Moreover, the inventors found that preparing multiple sequencing libraries from a single sample increases gene yield due to a sampling effect. The combination of methods steps described herein thus provides for the first time a method of producing a sequencing library from human mRNA in a stool sample, wherein the library comprises polynucleotide sequences complementary to at least about 3,000 human protein coding genes, at least about 4,000 human genes, at least about 5,000 human genes, at least about 5,000 human genes, at least about 6,000 human genes, at least about 7,000 human genes, at least about 8,000 human genes, at least about 9,000 human genes, or at least about 10,000 human genes.

Such methods, in combination with the sequence analysis workflows described herein have broad applications and provide unique advantages over currently available techniques. Furthermore, the disclosed methods provide an accurate, cost-effective platform technology enabling multi-omic longitudinal applications in deep phenotyping by combining gut (eukaryotic host) crosstalk with microbial (prokaryotic) responses to diet, therapeutics, and chronic disease.

### A. Producing a Sequencing Library from mRNA in a Stool Sample

The methods provided herein comprise producing a sequencing library from mRNA in a stool sample, such as human mRNA. In some embodiments, the method may comprise treating a stool sample with a stool stabilizing reagent, e.g. a stool stabilization reagent that inhibits degradation of polynucleotides. Stool stabilizing reagents, such as Zymo DNA/RNA Shield™, are formulated chemical solutions designed to preserve the integrity of nucleic acids and other molecular biomarkers in biological samples, under ambient conditions. These reagents function by inactivating enzymes, such as nucleases, which degrade nucleic acids and by maintaining the native state of the sample's microbiota, preventing shifts in microbial composition. The stabilization occurs immediately upon contact, eliminating the need for cold chain storage or immediate processing. Stool stabilizing reagents provide effective preservation by creating a chemically stable environment, which ensures the reliability of downstream molecular analyses, including genomic, transcriptomic, and metagenomic studies. Interestingly, no published research has been identified that utilizes Zymo DNA/RNA Shield for the preservation of mammalian RNA in stool samples. Accordingly, it is believed that the present disclosure is the first application of Zymo DNA/RNA Shield for mammalian stool RNA preparation, resulting in a multifold improvement in the number of detectable genes.

The methods provided herein further comprise isolating RNA from the stool sample treated with the stool stabilizing reagent based on the presence of a polyadenylated (polyA) tail. Stool samples are mixed biological samples comprising eukaryotic, bacterial, and viral nucleic acid sequences. The presence of a polyadenylated (polyA) tail, a characteristic of eukaryotic mRNA, can be leveraged to selectively capture and purify target host mRNA molecules. The process may involve introducing oligo (dT) probes, either immobilized on solid supports (e.g., magnetic beads) or in solution, which specifically hybridize with the polyA tails of mRNA under hybridization conditions. Non-target RNA species, such as ribosomal RNA (rRNA) and bacterial RNA lacking polyA tails, remain unbound and are removed through a series of washes. The bound mRNA is then eluted using conditions that disrupt the oligo (dT)-polyA interaction, resulting in a highly enriched mRNA fraction suitable for downstream applications, including cDNA synthesis, gene expression profiling, and transcriptome analysis. Using a polyA-based isolation and selection technique (after initial RNA isolation) as described herein, can significantly increase the number of sequence reads corresponding to host genes. In some embodiments, isolating RNA based on the presence of a polyadenylated (polyA) tail comprises use of a non-traditional oligo (dT)-type reagent. In particular embodiments, a polyT gripNA probe is used in the methods described herein, which has a higher affinity for mRNA, an ability to bind short poly A tails, and reduces non-specific binding of DNA and ribosomal RNA compared to traditional oligo (dT) probes. No published research has been identified that utilizes a polyT gripNA probe for RNA isolation from colon or stool. Accordingly, it is believed that the present disclosure is the first application of polyT gripNA for mammalian stool RNA isolation, resulting in a multifold improvement in the number of detectable genes. This selective isolation technique ensures efficient separation of mRNA from complex samples, enhancing sensitivity and accuracy in molecular analyses.

The methods provided herein further comprise obtaining a sample of isolated RNA comprising less than about 200 ng of polynucleotides. In this regard, it was surprisingly found that increasing the amount of mRNA used in the production of the sequencing library does not increase host gene yield. This may potentially be due to contaminants found in stool samples. Conversely, using a significant reduction in input RNA as compared to typical protocols, enables an enhanced gene output. In some embodiments, the sample of isolated RNA comprises less than about 200 ng of polynucleotides, less than about 190 ng of polynucleotides, less than about 180 ng of polynucleotides, less than about 175 ng of polynucleotides, less than about 170 ng of polynucleotides, less than about 165 ng of polynucleotides, less than about 160 ng of polynucleotides, less than about 155 ng of polynucleotides, less than about 150 ng of polynucleotides, less than about 140 ng of polynucleotides, less than about 130 ng of polynucleotides, less than about 125 ng of polynucleotides, less than about 120 ng of polynucleotides, less than about 115 ng of polynucleotides, less than about 110 ng of polynucleotides, less than about 100 ng of polynucleotides, less than about 90 ng of polynucleotides, less than about 80 ng of polynucleotides, or less than about 75 ng of polynucleotides, including all ranges derivable therebetween. In specific embodiments, the sample of the isolated RNA comprises about 100 ng of polynucleotides. As demonstrated herein, obtaining a sample of isolated RNA comprising less than about 200 ng of polynucleotides can increase the number of host gene sequencing reads by more than about 10%, more than about 15%, more than about 20%, more than about 25%, more than about 30%, more than about 35%, more than about 40%, more than about 15%, or more than about 50%, as compared to a sequencing library obtained from a sample of isolated RNA comprising more than 200 ng of polynucleotides, e.g. as compared to a sequencing library obtained from a sample of isolated RNA comprising about 300 ng or about 500 ng of polynucleotides.

Also provided herein are methods comprising reverse transcribing mRNA using polyA-targeting primers and incorporating a unique molecular identifier into the reverse transcribed mRNA. A Unique Molecular Identifier (UMI) is a short, random nucleotide sequence (e.g. 8-12 nucleotides) added to individual nucleic acid molecules at the beginning of a sequencing workflow to uniquely tag each original molecule. Incorporating unique molecular identifiers (UMIs) into reverse-transcribed mRNA libraries for sequencing involves the attachment of distinct, random nucleotide sequences to individual mRNA molecules during the reverse transcription process. The UMIs, within oligonucleotide primers, hybridize with the polyadenylated (polyA) tail of target mRNA and are reverse transcribed along with the RNA template, generating a complementary DNA (cDNA) molecule tagged with a unique sequence identifier. These UMIs serve as molecular barcodes, enabling the identification and differentiation of original RNA molecules from amplification artifacts. During downstream amplification and sequencing, reads with identical UMIs and alignment positions are treated as duplicates, ensuring that quantification reflects the actual abundance of transcripts rather than biases introduced during PCR. This helps improve the accuracy and sensitivity of transcriptomic analysis by minimizing amplification errors, enhancing the detection of low-abundance transcripts, and enabling precise gene expression quantification in complex or high-throughput sequencing workflows. As such, incorporating a unique molecular identifier into the reverse transcribed host mRNA molecules significantly increases data integrity, especially the quantitative measure of the genes expressed.

In related embodiments, the methods described herein comprise repeating the steps of obtaining a sample of isolated RNA comprising less than about 200 ng of polynucleotides, and reverse transcribing the human mRNA using polyA-targeting primers and incorporating a unique molecular identifier into the reverse transcribed human mRNA, to produce a plurality of sequencing libraries. The present inventors found that preparing multiple sequencing libraries from a single sample increases gene yield due to a sampling effect. That is, sampling can introduce bias or variability when a subset of polynucleotide molecules is randomly selected from a larger population for sequencing such as a sample of isolated RNA comprising less than about 200 ng of polynucleotides from a stool sample. Producing multiple sequencing libraries as described herein corrects for variability and ensures that the final sequencing data reflects the true biological composition as accurately as possible. Furthermore, such steps provide additional advantages including, but not limited to, increased sequencing depth and identification of low abundance sequences. In particular, producing multiple sequencing libraries as described herein from samples of isolated RNA comprising less than about 200 ng of polynucleotides significantly increases the number of unique host gene reads. For example, producing a plurality of sequencing libraries can result in sequencing reads corresponding to about 500 more host (e.g. human) genes as compared to a single sequencing library, about 500 more host genes, about 750 more host genes, about 1,000 more host genes, about 1,500 more host genes, about 2,000 more host genes, about 2,500 more host genes, about 3,000 more host genes, about 3,500 more host genes, about 4,000 more host genes, or about 5,000 more host genes, as compared to a single sequencing library. In other embodiments, the number of host genes detectable by the plurality of sequencing libraries relative to a single sequencing library may also be described as at least about 1.25-fold greater, as at least about 1.5-fold greater, as at least about 1.75-fold greater, as at least about 2-fold greater, as at least about 2.25-fold greater, as at least about 2.5-fold greater, as at least about 3-fold greater, as at least about 3.5-fold greater, as at least about 4-fold greater, or as at least about 5-fold greater. The methods steps provided herein may be repeated about two or more times, about three or more times, about four or more times, about five or more times, about six or more times, about seven or more times, about eight or more times, about nine or more times, or about ten or more times to produce a plurality of sequencing libraries. The sequencing libraries produced from the methods described herein may comprise polynucleotide sequences complementary to at least about 2,500 host protein coding genes, at least about 3,000 host protein coding genes, at least about 3,500 host protein coding genes, at least about 4,000 host protein coding genes, at least about 4,500 host protein coding genes, at least about 5,000 host protein coding genes, at least about 5,500 host protein coding genes, at least about 6,000 host protein coding genes, at least about 6,500 host protein coding genes, at least about 7,000 host genes, or at least about 7,500 host genes.

### B. Sequencing and Mapping

As used herein the term “primer” refers to a DNA molecule that is designed for use in annealing or hybridization methods that involve an amplification reaction. An amplification reaction is an in vitro reaction that amplifies template DNA or RNA to produce an amplicon. As used herein, an “amplicon” is a DNA molecule that has been synthesized using amplification techniques. A pair of primers may be used with template DNA or RNA, such as a sample of host mRNA, in an amplification reaction, such as polymerase chain reaction (PCR), to produce an amplicon, where the amplicon produced would have a DNA sequence corresponding to sequence of the template DNA or RNA located between the two sites where the primers hybridized to the template. A primer is typically designed to hybridize to a complementary target DNA strand to form a hybrid between the primer and the target cDNA strand. The presence of a primer is a point of recognition by a polymerase to begin extension of the primer using as a template the target DNA or RNA strand. Primer pairs refer to use of two primers binding opposite strands of a double stranded nucleotide segment for the purpose of amplifying the nucleotide segment between them.

The amplified fragments may be used for high-throughput sequencing. In some embodiments, the PCR primers used to amplify the cDNA fragments comprise sequencing adaptors used for high-throughput sequencing. Methods and primers for high-throughput sequencing are known in the art and any such methods or primers may be used according to the methods of the present disclosure. Non-limiting examples of which include next generation sequencing, single molecule sequencing, and nanopore sequencing.

The present disclosure further provides a method of detecting the presence of a human gene sequence expressed in a human gut, comprising sequencing a plurality of sequencing libraries produced by the methods described herein; and mapping the sequences produced using a computer algorithm. “Mapping the sequences” or “Sequence mapping” as used herein involves the computational alignment of raw sequencing reads to a reference genome or transcriptome to identify the origin and structure of each sequenced fragment. Sequence mapping begins by obtaining raw reads from sequencing platforms in a digital format, such as FASTQ, which contain both nucleotide sequences and associated quality scores. Pre-processing steps may include adapter trimming, quality filtering, and removal of low-complexity sequences to optimize alignment accuracy. The resulting sequence reads are then aligned against a reference genome or transcriptome using algorithms such as Burrows-Wheeler Transform (BWT) or hash-based indexing methods, which facilitate rapid searching and alignment of reads to known genomic locations. During the alignment, mismatches, insertions, and deletions (indels) are identified and tolerated within a pre-defined threshold optimized for read lengths and error modes yielded by typical Illumina sequencers. An example sequence mapping program for use in the methods described herein may include Bowtie2, which is effective for aligning short reads produced by high-throughput sequencing technologies. Typically, pre-defined thresholds do not account for the biological variation due to transcript degradation and/or lower quality reads, and require adjustment to improve sequence alignment

Bowtie2 uses the Burrows-Wheeler Transform (BWT) and FM-index to compress reference genomes and perform rapid searches, making it highly efficient even with large reference genomes. In some embodiments, the computer algorithm, such as Bowtie2, used in the disclosed methods, comprises an adjusted mismatch penalty or an adjusted match bonus setting. The mismatch penalty setting, and match bonus setting are scoring parameters that influence how reads are aligned to a reference genome, which determine the overall score of an alignment and thus whether a particular alignment is valid or optimal. A higher mismatch penalty discourages mismatches, making the aligner more stringent. This can result in fewer mismatches but may cause valid alignments with some natural variation (e.g., SNPs) to be missed; whereas a lower mismatch penalty allows for more mismatches, making the alignment process more permissive, which may be useful for aligning reads from highly variable regions. Regarding the match bonus setting, a higher match bonus increases the alignment score for matching bases, encouraging the aligner to prioritize alignments with a high number of exact matches and thus improves sensitivity. Lowering the mismatch penalty from the pre-determined threshold (6) to 4 or 2, allows reads to have more variation and makes the alignment process more permissive for lower quality transcripts. By increasing the match bonus from the pre-determined threshold (2) to 6 or 8, the aligner prioritizes the sequence alignments to include exact matches. Each new dataset requires benchmarking to determine the best value for each setting In certain embodiments, mapping the sequences results in an alignment rate of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.

### C. Applications of Sequencing Results Derived from Stool Samples

Using the methods described herein, a sequencing library may be produced from practically any eukaryotic organism stool sample. Non-limiting examples of such organisms include animals, mammals, and humans. In particular embodiments, the organism may include a human (adult and/or infant at any stage of development), a mouse, a horse, or a pig. In specific embodiments, the human stool sample may be from an infant, such as a preterm infant, about a 2 week old infant, about a 4 week old infant, about a 8 week old infant, about a 12 week old infant, about a 4 month old infant, about a 5 month old infant, about a 6 month old infant, about a 12 month old infant, or about a 4 year old infant. The methods described herein provide the ability to isolate, process, analyze and interpret nutritional and clinical predictors of intestinal maturation and feeding tolerance in preterm infants. For example, a sequencing library or plurality thereof can be produced and analyzed following a dietary intervention or medical intervention. As such, the disclosed methods allow clinicians to identify eukaryotic gene expression changes in response to a given intervention and direct treatment decisions based on the same. For example, provided herein is a method comprising detecting the presence of a decreased or increased level of a nutrient absorption gene mRNA relative to a control, such as the level of SLC1A1, SLC38A1, SLC38A2, ABCG5, SLC26A2, LPL, SAR1B, SLC44A1, or BTD mRNA relative to an appropriate control. Such detection would, e.g. inform a clinician regarding whether the infant has developed any nutrient absorption/transport function-related deficiencies. As such, the clinician may treat the infant by administering a dietary supplement or altering a dietary supplementation strategy to accommodate the biochemical defect. In other embodiments, the methods provided herein may comprise detecting the presence of a decreased or increased level of a barrier function gene, a hypoxia-related gene, a GI ischemia-related gene, a heat shock protein gene, a HDAC response gene, a butyrate metabolism gene, an energy utilization gene, a stemness gene, or an immune response gene mRNA relative to an appropriate control. For example, in some embodiments, provided herein is a method comprising detecting the presence of a decreased or increased level of an immune response gene mRNA relative to a control. In certain embodiments, the method comprises detecting the presence of a decreased or increased level of ADCY1, JAK1, RPL30, TRIM14, CCL25, CD180, CD1D, CFHR1, COLEC11, CST9, CXCR4, ECM1, IFIT1B, IFNA21, IL4, MASP2, MBL2, NMB, or PGLYRP3 mRNA relative to an appropriate control. In further embodiments, the method comprises detecting the presence of a decreased level of ADCY1, JAK1, RPL30, or TRIM14 mRNA relative to an appropriate control; or an increased level of CCL25, CD180, CD1D, CFHR1, COLEC11, CST9, CXCR4, ECM1, IFIT1B, IFNA21, IL4, MASP2, MBL2, NMB, or PGLYRP3 mRNA relative to an appropriate control. In some embodiments, the method comprises detecting the presence of a decreased or increased level of AATBC, ABCA1, ABLIM1, AC024600.1, AC078883.2, ADCY1, ADGRV1, AGO4, AJAP1, AL591135.1, ANKS1A, ARFGEF3, ASAP1, ATP2B1-AS1, BEND3, CA13, CCDC50, CCDC85C, CIPC, COL25A1, CSNK1G2, CTSB, DNAJC5, EIF2S2P2, EXOSC6, FRYL, GABRA2, GALNT9, GON4L, GRWD1, GTF3A, HEG1, HES1, HIVEP3, HMCN1, HOXA10, IL6ST, JAK1, KCNJ14, KIAA0232, LAMB1, LINC01187, LRRC37BP1, MLXIPL, MOCS2, MPP5, MPV17, MSL1, MYO5B, NDUFA5, NDUFB9, NEAT1, NEU1, NIPAL1, NUDCD2, OTUD1, OTUD3, PACSIN3, PARD3B, PLEKHG2, PPM1K, PPP1CB, PPP3R1, PRMT8, PTPN12, PVR, RABL6, RALBP1, RALGDS, REST, RNF157, RNF34, RPL30, SCAMP4, SFI1, SIKE1, SMPD3, SP2, SPIRE2, TEAD3, TNIK, TOMM20, TRIM14, TRUB2, TTC19, UBE3A, VAV2, YIPF5, ZFR, ZNF570, ZNF701, ZNF706, ZNF791, ZNHIT1, AC007491.1, AC018553.2, AC064799.2, AC090844.2, AC129502.1, AL513190.1, AL590235.1, AP1B1P1, BX255925.1, C5orf66-AS2, CD200RIL, COL4A6, EFHC2, EXOG, GLIDR, KCNF1, LINC01960, MASP2, MROH4P, OXLD1, or SNPH mRNA relative to an appropriate control. In specific embodiments, the method comprises detecting the presence of a decreased level of AATBC, ABCA1, ABLIM1, AC024600.1, AC078883.2, ADCY1, ADGRV1, AGO4, AJAP1, AL591135.1, ANKS1A, ARFGEF3, ASAP1, ATP2B1-AS1, BEND3, CA13, CCDC50, CCDC85C, CIPC, COL25A1, CSNK1G2, CTSB, DNAJC5, EIF2S2P2, EXOSC6, FRYL, GABRA2, GALNT9, GON4L, GRWD1, GTF3A, HEG1, HES1, HIVEP3, HMCN1, HOXA10, IL6ST, JAK1, KCNJ14, KIAA0232, LAMB1, LINC01187, LRRC37BP1, MLXIPL, MOCS2, MPP5, MPV17, MSL1, MYO5B, NDUFA5, NDUFB9, NEAT1, NEU1, NIPAL1, NUDCD2, OTUD1, OTUD3, PACSIN3, PARD3B, PLEKHG2, PPM1K, PPP1CB, PPP3R1, PRMT8, PTPN12, PVR, RABL6, RALBP1, RALGDS, REST, RNF157, RNF34, RPL30, SCAMP4, SFI1, SIKE1, SMPD3, SP2, SPIRE2, TEAD3, TNIK, TOMM20, TRIM14, TRUB2, TTC19, UBE3A, VAV2, YIPF5, ZFR, ZNF570, ZNF701, ZNF706, ZNF791, or ZNHIT1 mRNA relative to an appropriate control; or an increased level of AC007491.1, AC018553.2, AC064799.2, AC090844.2, AC129502.1, AL513190.1, AL590235.1, AP1B1P1, BX255925.1, C5orf66-AS2, CD200RIL, COL4A6, EFHC2, EXOG, GLIDR, KCNF1, LINC01960, MASP2, MROH4P, OXLD1, or SNPH mRNA relative to an appropriate control.

In some embodiments, the increased level of the mRNA may also be described as at least about 1.25-fold greater, as at least about 1.5-fold greater, as at least about 1.75-fold greater, as at least about 2-fold greater, as at least about 5-fold greater, as at least about 10-fold greater, as at least about 25-fold greater, as at least about 50-fold greater, as at least about 100-fold greater, or as at least about 150-fold greater relative to an appropriate control. Similarly, the decreased level of the mRNA may also be described as at least about 1.25-fold less, as at least about 1.5-fold less, as at least about 1.75-fold less, as at least about 2-fold less, as at least about 5-fold less, as at least about 10-fold less, as at least about 25-fold less, as at least about 50-fold less, as at least about 100-fold less, or as at least about 150-fold less relative to an appropriate control.

The methods provided herein allow for accurately analyzing gastrointestinal health through non-invasive methods. Furthermore, utilizing data from exfoliated epithelial cells collected from stool as described herein, combined with microbiome sequencing, enables personalized predictions about how diet, therapeutics, and/or chronic disease, and microbial interactions, affect intestinal health and development, without the need for invasive biopsies or tissue samples. This framework offers the potential to explore the synergy between host gene expression and microbial communities, and predict individualized responses to therapies. By integrating transcriptomic and microbial data, these methods can uncover molecular mechanisms that influence gut health and development at an individual level. The methods provided herein also allow for longitudinal analysis. For example, monitoring the expression levels of individual or combinations of genes from the same subject in order to assess changes due to development or effects of a treatment or condition.

These investigations and the directed treatments derived therefrom would not be feasible without the methods disclosed herein. The ability to evaluate complex, multivariate relationships between the host transcriptome and microbiome (e.g., stool derived 16S rRNA, shotgun DNA sequencing, metabolome) “multi-omic” applications significantly advances personalized medicine. In regards to human infants, this approach lays the foundation for predicting whether certain infants will benefit more from specific interventions, such as breastfeeding or formula feeding or specialized formulas/diets, ensuring superior clinical outcomes through precision nutrition and personalized health monitoring.

### C. Informed Treatment Decisions

The methods provided herein allow for treating a patient based on an increased or decreased level of an mRNA relative to an appropriate control. For example, provided herein is a method of treating an infant with feeding intolerance comprising obtaining a sample from the infant that comprises at least a first mRNA associated with feeding intolerance; detecting in the sample the presence of an increased or decreased level of the mRNA relative to a control infant that lacks feeding intolerance; and treating the infant with feeding intolerance based on the increased or decreased level of the mRNA relative to the control. In some embodiments, treating the infant comprises no treatment. In other embodiments, treating the infant comprises administering intravenous nutrition. In specific embodiments, the method comprises administering a partially hydrolyzed formula or human milk treated with enzymes. Similarly, treatment decisions can be informed using the methods described herein related to preterm delivery, GI ischemia, small bowel resection, or damage to the intestinal absorptive surface due to infection or drugs (e.g., chemotherapeutic) or radiation therapy. Thus, provided herein is a method of treating a patient exhibiting any of these conditions, comprising obtaining a sample from the patient that comprises at least a first mRNA associated with the condition or disease; detecting in the sample the presence of an increased or decreased level of the mRNA relative to a control patient that lacks the condition or disease; and treating the patient based on the increased or decreased level of the mRNA relative to the control. Regarding GI ischemia, in some embodiments, treating the patient comprises administering using intravenous nutrition or administering human milk treated with enzymes, partially hydrolyzed formula, or formulas with more easily digested and absorbed components (e.g., medium chain triglycerides). In some embodiments, such treatment decisions can be based on the expression levels of a combination or one or more, two or more, three or more, four or more, five or more, six or more, seven or more, eight or more, nine or more, or ten or more, genes.

### D. Kits

In certain aspects, the present disclosure provides kits that may be used for performing the methods provided by the present disclosure. In some embodiments, such kits may comprise one or more of the following: a stool stabilizing reagent, polyT gripNA probe, a lysis buffer, a wash buffer, an elution buffer, oligo (dT) primers and UMI sequences, a reverse transcriptase enzyme, dNTPs, a neutralization buffer. In another embodiment, the kit may further comprise instructions for use of the kit.

### E. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and genomics.

When introducing elements of the present disclosure or the embodiment(s) thereof, the articles “a,” “an,” “the,” and “said” are intended to mean that there are one or more of the elements.

The term “and/or,” when used in a list of two or more items, means any one of the items, any combination of the items, or all of the items with which this term is associated.

The terms “comprising,” “including,” and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “human” includes a person at any stage of development.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### Examples

The following examples are included to illustrate embodiments of the present disclosure. It should be appreciated by those of skill in the art that the techniques disclosed in the examples that follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1: Sequence Library Preparation from Human Stool

The following example describes the production of a sequencing library from human mRNA in a stool sample.

Stool samples were obtained, and small aliquots of fresh stool were placed into vials prefilled with DNA/RNA Shield (Zymo Research), homogenized to make a uniform slurry and frozen at −80° C. until further processing. RNA was isolated using polyT gripNA probes to specifically enrich for polyadenylated RNA (mRNA), followed by treatment with DNase to remove any contaminating DNA. Multiple RNA sequencing libraries were constructed from each sample with less than or equal to 200 ng of isolated stool RNA for each library, using oligo (dT) primers and incorporating universal molecular identifiers. Libraries were pooled and sequenced with standard protocols. Sequencing data are aligned to the human reference genome using settings determined by benchmarking in Bowtie2.

### Example 2: Gut Microbial and Host Intestinal Cell Response to a Fish Oil/Soluble Corn Fiber Supplement

The differential expression (DE) of genes in the glucose-insulin receptor-phosphatidylinositol (PI)-3 kinase signaling axis in response to a dietary supplement combining fish oil and soluble corn fiber will be investigated, in comparison to a supplement of corn oil and maltodextrin in older healthy individuals. It is expected that these studies will demonstrate that the insulin-PI3K signaling axis, as well as the AKT pathway, in the gut are suppressed by the fish oil/soluble corn fiber intervention, which is noteworthy because the insulin-PI3K-AKT pathway can drive malignant transformation. The predicted inhibition of pro-inflammatory genes in this intervention, e.g., NFKB1, IFNG, INFB, IL4, PRKAA1, and STAT3, would be consistent with studies showing that consumption of fish oil and fermentable fiber reduces chronic inflammatory markers, which may alter pathways involved in carcinogenesis. The insights from this study will provide a foundation for future research focused on dietary interventions for CRC prevention, emphasizing the importance of diet-gut microbiome interactions.

### Example 3: Identification of Novel Indicators of Neonatal Intestinal Development and Health

In 2020, 10% of U.S. infants were born preterm and ˜2%, or 60,000 infants, were born very preterm (VPI; <32 weeks PMA). VPI infants are at high risk for of substantial medical complications, including necrotizing enterocolitis (NEC). In VPI, advancing and maintaining nutritional support reduces disease risk and improves neurodevelopmental outcomes; however, up to 25% of preterm infants demonstrate feeding intolerance, which may be benign or may progress to NEC.

Up to 6 stool samples were collected from very preterm infants (VPIs) enrolled in a longitudinal prospective cohort between birth and 36 wk postmenstrual age. VPIs who have demonstrated feeding intolerance, or intestinal ischemia were selected for analysis and demographically matched to infants without any intestinal concerns. The first objective was to determine exfoliated intestinal cell RNA yield in VPI and compare genomic biomarkers to other populations. Host cell RNA was isolated from stool samples and sequenced using the Illumina NovaSeq X Plus platform according to the methods described herein in order to accurately assess the host intestinal transcriptome.

Preliminary sequencing results from 9 infants demonstrated over 100 million reads each, and the full sequencing depth possible was achieved. Between 6,800 and 11,000 host genes were detected in the VPIs exfoliome. The mean number of genes in the exfoliome of VPIs (8,884) was similar to 2-week-old term infants (10,495) and higher than that of 5-month-old term infants (5,951), 4-year-old children (2,976), and adults (3,013). Gene biomarkers were used to identify cell types and functions in the small and large intestine. In addition, heat maps of the average counts of nutrient absorption genes, including amino acid, bile acid, inorganic solute, lipid, metal ion, and nucleotides, were generated. Patterns of gene expression in VPIs were more like those in term infants than in children. Further analyses was focused on gene biomarkers and patterns that differentiate infants with feeding intolerance, intestinal ischemia, and those without intestinal issues. Table 1 lists the immune response genes that were expressed in at least 1 of the NEC babies without appearing in the healthy infants; and the immune response genes that were expressed in all healthy infants without appearing in the NEC babies. Similarly, Table 2 lists all genes that were expressed in at least I of the NEC babies without appearing in the healthy infants; and all genes that were expressed in all healthy infants without appearing in the NEC babies.

TABLE 1
Immune Response gene expression in
non-NEC vs NEC premature infants.

| | |
|---|---|
| Immune Response | ADCY1, JAK1, RPL30, TRIM14 |
| Genes that were | |
| expressed in all healthy | |
| infants without appearing | |
| in the NEC babies | |
| Immune Response Genes that were | CCL25, CD180, CD1D, CFHR1, |
| expressed in at least 1 | COLEC11, CST9, CXCR4, ECM1, |
| of the NEC babies without | IFIT1B, IFNA21, IL4, MASP2, |
| appearing in the healthy infants | MBL2, NMB, PGLYRP3 |

TABLE 2
Gene expression in non-NEC vs NEC premature infants (all genes).

| | |
|---|---|
| Genes that were expressed in all healthy | AATBC, ABCA1, ABLIM1, AC024600.1, |
| infants without appearing in the NEC | AC078883.2, ADCY1, ADGRV1, AGO4, AJAP1, |
| babies | AL591135.1, ANKS1A, ARFGEF3, ASAP1, |
| | ATP2B1-AS1, BEND3, CA13, CCDC50, |
| | CCDC85C, CIPC, COL25A1, CSNK1G2, CTSB, |
| | DNAJC5, EIF2S2P2, EXOSC6, FRYL, GABRA2, |
| | GALNT9, GON4L, GRWD1, GTF3A, HEG1, |
| | HES1, HIVEP3, HMCN1, HOXA10, IL6ST, JAK1, |
| | KCNJ14, KIAA0232, LAMB1, LINC01187, |
| | LRRC37BP1, MLXIPL, MOCS2, MPP5, MPV17, |
| | MSL1, MYO5B, NDUFA5, NDUFB9, NEAT1, |
| | NEU1, NIPAL1, NUDCD2, OTUD1, OTUD3, |
| | PACSIN3, PARD3B, PLEKHG2, PPM1K, PPP1CB, |
| | PPP3R1, PRMT8, PTPN12, PVR, RABL6, |
| | RALBP1, RALGDS, REST, RNF157, RNF34, |
| | RPL30, SCAMP4, SFI1, SIKE1, SMPD3, SP2, |
| | SPIRE2, TEAD3, TNIK, TOMM20, TRIM14, |
| | TRUB2, TTC19, UBE3A, VAV2, YIPF5, ZFR, |
| | ZNF570, ZNF701, ZNF706, ZNF791, ZNHIT1 |
| Genes that were expressed in at least 1 | AC007491.1, AC018553.2, AC064799.2, |
| of the NEC babies without appearing in | AC090844.2, AC129502.1, AL513190.1, |
| the healthy infants | AL590235.1, AP1B1P1, BX255925.1, C5orf66- |
| | AS2, CD200R1L, COL4A6, EFHC2, EXOG, |
| | GLIDR, KCNF1, LINC01960, MASP2, MROH4P, |
| | OXLD1, SNPH |

The methods of producing sequencing libraries described herein allow for accurate analysis of gastrointestinal health through non-invasive methods. In this case, specifically providing means to better understand the gene networks and metabolic pathways driving intestinal disease in VPI. For example, using the methods described herein the following differentially expressed genes in non-NEC (n=6) vs NEC (n=3) premature infants with p-value<0.05 were detected.

TABLE 3
Differentially expressed genes in non-NEC (n =
6) vs NEC (n = 3) premature infants.

| Gene Name | Fold Change | Pvalue |
|---|---|---|
| SEC63 | 0.0709 | 0.0002 |
| HNI13 | 0.0555 | 0.0003 |
| RALBP1 | 49.4324 | 0.0004 |
| CTSB | 99.2582 | 0.0014 |
| AC007491.1 | 0.1114 | 0.0015 |
| NEAT1 | 99.4759 | 0.0018 |
| RAB11B | 0.0878 | 0.0023 |
| RABL6 | 19.3921 | 0.0024 |
| HSP90AA1 | 201.4181 | 0.0025 |
| JUND | 72.3339 | 0.0028 |
| ZFP36L1 | 60.1797 | 0.0034 |
| SLC5A12 | 187.9874 | 0.0037 |
| TRINI38 | 38.3401 | 0.0037 |
| HSP90AA2P | 126.0447 | 0.0037 |
| SRRNI2 | 30.9088 | 0.0038 |
| LPP | 25.2546 | 0.0038 |
| SLC35A3 | 21.4673 | 0.004 |
| ZNF740 | 13.834 | 0.004 |
| ARFGEF3 | 13.9443 | 0.0043 |
| NIBOAT2 | 0.1511 | 0.0043 |
| RNFT2 | 17.5293 | 0.0044 |
| TTC39B | 73.0548 | 0.0044 |
| KLF6 | 34.2988 | 0.0057 |
| AC012186.2 | 0.1214 | 0.006 |
| RBNI47 | 119.614 | 0.007 |
| PTNIAP2 | 53.895 | 0.0077 |
| PAX6 | 13.977 | 0.008 |
| NIKNK2 | 0.1842 | 0.0083 |
| CANIK2N1 | 40.31 | 0.0091 |
| TRIO | 12.4102 | 0.0103 |
| PTNIAP5 | 39.1311 | 0.0104 |
| GK5 | 8.3867 | 0.0111 |
| RSRC2 | 16.2133 | 0.0112 |
| SANIHD1 | 48.4685 | 0.0115 |
| PYY2 | 0.1468 | 0.0117 |
| CDK13 | 15.3111 | 0.012 |
| AL365440.1 | 22.7338 | 0.0123 |
| PRRC2C | 26.2117 | 0.014 |
| SCN3A | 0.3052 | 0.0145 |
| LINC00554 | 0.1516 | 0.0148 |
| GPRC5A | 13.5157 | 0.0155 |
| GLIPR1 | 0.2712 | 0.0159 |
| TENT5A | 14.558 | 0.016 |
| PRRG4 | 17.0162 | 0.016 |
| ADAP1 | 43.5902 | 0.0162 |
| AC 103691.1 | 60.6453 | 0.0184 |
| SATB1-AS1 | 0.1609 | 0.021 |
| AC020916.1 | 8.9017 | 0.021 |
| NIAF | 4.1921 | 0.0216 |
| ERBIN | 10.326 | 0.0219 |
| NILLT6 | 9.3219 | 0.0224 |
| RBNI25 | 18.3467 | 0.0224 |
| RAB21 | 8.217 | 0.0226 |
| GSN | 16.7728 | 0.0231 |
| NIALAT1 | 30.467 | 0.0231 |
| KIF3B | 16.3272 | 0.0234 |
| PHF12 | 0.1877 | 0.0247 |
| THOC2 | 11.0156 | 0.0249 |
| RCAN1 | 20.0803 | 0.0252 |
| RRBP1 | 15.1363 | 0.0252 |
| UBTFL9 | 0.1833 | 0.0253 |
| ZBED3-AS1 | 0.1605 | 0.0255 |
| FZD2 | 0.1738 | 0.0265 |
| GEN1 | 0.1656 | 0.0273 |
| LRIG2 | 0.1945 | 0.0308 |
| RNF213 | 9.1686 | 0.0314 |
| NIROH7 | 20.7272 | 0.0324 |
| DCUN1D1 | 0.2861 | 0.0325 |
| NCOR1 | 8.0144 | 0.0363 |
| NITRNR2L12 | 17.7565 | 0.0369 |
| ANP32BP1 | 15.152 | 0.0369 |
| KCNK6 | 13.31 | 0.0377 |
| KLHL24 | 5.6485 | 0.0384 |
| SCAF11 | 9.2783 | 0.0394 |
| NCALD | 15.3163 | 0.0398 |
| AC092910.3 | 54.5555 | 0.0403 |
| FGD2 | 8.3894 | 0.041 |
| PABPN1 | 5.5984 | 0.0422 |
| AEN | 0.2448 | 0.0423 |
| SOX4 | 4.8691 | 0.044 |
| ARPC2 | 6.1652 | 0.0443 |
| RASSF3 | 6.0435 | 0.0446 |
| NIETTL21A | 0.1993 | 0.0457 |
| PEBP1 | 0.2126 | 0.0461 |
| AC092376.2 | 0.2486 | 0.0483 |
| ZNF84 | 11.8171 | 0.0487 |
| NICOLN3 | 0.2512 | 0.0499 |

Furthermore, Ingenuity Pathway Analysis (IPA) can be used to map differentially expressed genes onto known biological pathways and interaction networks to identify affected biological processes. Here, differentially expressed genes with p<0.01 were used for Ingenuity Pathway Analysis (IPA) of upstream regulators as well as diseases and functions. The identified upstream regulators and diseases and functions are shown in Tables 2 and 3 below, respectively. Upstream regulators/Diseases and functions with activation z-score>0 are trending toward activation and those with an activation z-score<0 are trending toward inhibition in non-NEC babies compared to NEC babies.

TABLE 4
Differentially expressed genes with p < 0.01
were used for IPA analysis of upstream regulators.

| Upstream | Molecule | p-value of | z- | Predicted | Target Molecules in |
|---|---|---|---|---|---|
| Regulator | Type | overlap | score | Activation | Dataset |
| gentamicin | CD | 3.60E−04 | 2.0 | Increased | CTSB |
| | | | | | HSP90AA1 |
| | | | | | KLF6 |
| | | | | | ZFP36L1 |
| NUPR1 | TR | 1.43E−03 | 1.0 | | CAMK2N1 |
| | | | | | KLF6 |
| | | | | | RNFT2 |
| | | | | | ZFP36L1 |
| tretinoin | CD | 8.92E−03 | 1.0 | | CTSB |
| | | | | | HSP90AA1 |
| | | | | | JUND |
| | | | | | PAX6 |
| | | | | | RAB11B |
| | | | | | ZFP36L1 |
| lipopolysaccharide | CD | 7.36E−03 | 0.9 | | CTSB |
| | | | | | HM13 |
| | | | | | HSP90AA1 |
| | | | | | JUND |
| | | | | | KLF6 |
| | | | | | MKNK2 |
| | | | | | NEAT1 |
| | | | | | TTC39B |
| APP | O | 5.17E−04 | 0.8 | | CTSB |
| | | | | | HSP90AA1 |
| | | | | | JUND |
| | | | | | NEAT1 |
| | | | | | PAX6 |
| | | | | | ZFP36L1 |
| IFNG | CK | 5.74E−03 | 0.7 | | CTSB |
| | | | | | HSP90AA1 |
| | | | | | JUND |
| | | | | | KLF6 |
| | | | | | NEAT1 |
| | | | | | PAX6 |
| TP53 | TR | 1.90E−03 | 0 | | CAMK2N1 |
| | | | | | CTSB |
| | | | | | HSP90AA1 |
| | | | | | JUND |
| | | | | | KLF6 |
| | | | | | LPP |
| | | | | | RALBP1 |
| | | | | | ZFP36L1 |
| TGFB1 | GF | 1.59E−02 | −0.2 | | CAMK2N1 |
| | | | | | CTSB |
| | | | | | HSP90AA1 |
| | | | | | JUND |
| | | | | | MBOAT2 |
| | | | | | MKNK2 |
| dexamethasone | CD | 2.88E−02 | −0.6 | | CTSB |
| | | | | | JUND |
| | | | | | KLF6 |
| | | | | | PAX6 |
| | | | | | SEC63 |
| | | | | | ZFP36L1 |
| IL1B | CK | 3.81E−02 | −0.7 | | CTSB |
| | | | | | JUND |
| | | | | | NEAT1 |
| | | | | | PAX6 |

CD = chemical drug;
TR = transcription regulator;
O = other,
CK = cytokine;
GF = growth factor

TABLE 5
Differentially expressed genes with p < 0.01
were used for IPA analysis of diseases and functions.

| Categories Diseases | | | | | |
|---|---|---|---|---|---|
| or Functions | | Predicted | | | # |
| Annotation | p-value | Activation | z-score | Molecules | Molecules |
| Inflammatory Response | 0.0119 | Increased | 2.0 | CAMK2N | 5 |
| | | | | CTSB | |
| | | | | HSP90AA1 | |
| | | | | JUND | |
| | | | | NEAT1 | |
| Infectious Diseases, | 0.000287 | | 1.2 | ARFGEF3 | 12 |
| Organismal Injury and | | | | CTSB | |
| Abnormalities, Viral | | | | HSP90AA1 | |
| Infection | | | | KLF6 | |
| | | | | LPP | |
| | | | | RAB11B | |
| | | | | RNFT2 | |
| | | | | SLC35A3 | |
| | | | | SLC5A12 | |
| | | | | SRRM2 | |
| | | | | TRIM38 | |
| | | | | TTC39B | |
| Cellular Function and | 0.0233 | | 1.2 | ARFGEF3 | 7 |
| Maintenance, Cellular | | | | CTSB | |
| homeostasis | | | | HSP90AA1 | |
| | | | | NEAT1 | |
| | | | | PAX6 | |
| | | | | RBM47 | |
| | | | | ZFP36L1 | |
| Molecular Transport | 0.00282 | | 1.1 | HSP90AA1 | 8 |
| | | | | JUND | |
| | | | | RAB11B | |
| | | | | RALBP1 | |
| | | | | SLC35A3 | |
| | | | | SLC5A12 | |
| | | | | TTC39B | |
| | | | | ZFP36L1 | |
| Infectious Diseases, | 0.00727 | | 1.1 | HSP90AA1 | 4 |
| Organismal Injury and | | | | RAB11B | |
| Abnormalities, | | | | SRRM2 | |
| Replication of RNA | | | | TRIM38 | |
| virus | | | | | |
| Cellular Development, | 0.000614 | | 1.0 | MKNK2 | 4 |
| Cellular Growth and | | | | PAX6 | |
| Proliferation, | | | | RABL6 | |
| Proliferation of | | | | RALBP1 | |
| pancreatic cancer cell | | | | | |
| lines | | | | | |
| Cellular Development, | 0.00733 | | 0.9 | HSP90AA1 | 4 |
| Cellular Growth and | | | | KLF6 | |
| Proliferation, Cell | | | | RABL6 | |
| proliferation of | | | | RALBP1 | |
| colorectal cancer cell | | | | | |
| lines | | | | | |
| Cellular Development, | 0.000123 | | 0.9 | L1 | 13 |
| Cellular Growth and | | | | | |
| Proliferation, Cell | | | | | |
| proliferation of tumor | | | | | |
| cell lines | | | | | |
| Inflammatory | 0.00677 | | 0.9 | CTSB | 10 |
| Response, Organismal | | | | HSP90AA1 | |
| Injury and | | | | LPP | |
| Abnormalities, | | | | NEAT1 | |
| Inflammation of | | | | PAX6 | |
| absolute anatomical | | | | RBM47 | |
| region | | | | SLC35A3 | |
| | | | | TRIM38 | |
| | | | | TTC39B | |
| | | | | ZFP36L1 | |
| Cellular Movement, | 0.0196 | | 0.8 | CTSB | 4 |
| Invasion of carcinoma | | | | HSP90AA1 | |
| cell lines | | | | JUND | |
| | | | | NEAT1 | |
| Inflammatory | 0.0138 | | 0.6 | CTSB | 4 |
| Response, Immune | | | | HSP90AA1 | |
| response of cells | | | | RALBP1 | |
| | | | | TRIM38 | |
| Cellular Movement | 0.00186 | | 0.5 | CAMK2N1 | 12 |
| | | | | CTSB | |
| | | | | HSP90AA1 | |
| | | | | JUND | |
| | | | | KLF6 | |
| | | | | LPP | |
| | | | | NEAT1 | |
| | | | | PAX6 | |
| | | | | RALBP1 | |
| | | | | RBM47 | |
| | | | | SLC35A3 | |
| | | | | ZFP36L1 | |
| Cell Cycle, Senescence | 0.000149 | | 0.4 | HM13 | 5 |
| of cells | | | | HSP90AA1 | |
| | | | | JUND | |
| | | | | KLF6 | |
| | | | | ZFP36L1 | |
| Cellular Movement, | 0.00335 | | 0.3 | CAMK2N1 | 11 |
| Migration of cells | | | | CTSB | |
| | | | | HSP90AA1 | |
| | | | | KLF6 | |
| | | | | LPP | |
| | | | | NEAT1 | |
| | | | | PAX6 | |
| | | | | RALBP1 | |
| | | | | RBM47 | |
| | | | | SLC35A3 | |
| | | | | ZFP36L1 | |
| Cellular Movement, | 0.00605 | | 0.3 | CTSB | 8 |
| Invasion of cells | | | | HSP90AA1 | |
| | | | | JUND | |
| | | | | KLF6 | |
| | | | | LPP | |
| | | | | NEAT1 | |
| | | | | RALBP1 | |
| | | | | RBM47 | |
| Cellular Movement, | 0.0135 | | 0.2 | CTSB | 7 |
| Invasion of tumor cell | | | | HSP90AA1 | |
| lines | | | | JUND | |
| | | | | KLF6 | |
| | | | | LPP | |
| | | | | NEAT1 | |
| | | | | RBM47 | |
| Cellular Development, | 0.00171 | | 0.1 | CTSB | 6 |
| Cellular Growth and | | | | JUND | |
| Proliferation, Colony | | | | KLF6 | |
| formation of cells | | | | MKNK2 | |
| | | | | NEAT1 | |
| | | | | PAX6 | |
| Cellular Development, | 0.0223 | | 0.0 | HSP90AA1 | 5 |
| Cellular Growth and | | | | KLF6 | |
| Proliferation, Cell | | | | NEAT1 | |
| proliferation of | | | | RALBP1 | |
| carcinoma cell lines | | | | RBM47 | |
| Cancer, Organismal | 0.00106 | | 0.0 | ARFGEF3 | 25 |
| Injury and | | | | CAMK2N1 | |
| Abnormalities | | | | CTSB | |
| Extracranial solid | | | | HM13 | |
| tumor | | | | HSP90AA1 | |
| | | | | JUND | |
| | | | | KLF6 | |
| | | | | LPP | |
| | | | | MBOAT2 | |
| | | | | MKNK2 | |
| | | | | NEAT1 | |
| | | | | PAX6 | |
| | | | | RAB11B | |
| | | | | RABL6 | |
| | | | | RALBP1 | |
| | | | | RBM47 | |
| | | | | RNFT2 | |
| | | | | SEC63 | |
| | | | | SLC35A3 | |
| | | | | SLC5A12 | |
| | | | | SRRM2 | |
| | | | | TRIM38 | |
| | | | | TTC39B | |
| | | | | ZFP36L1 | |
| | | | | ZNF740 | |
| Gastrointestinal | 0.00149 | | −0.1 | ARFGEF3 | 17 |
| Disease, Hepatic | | | | CAMK2N1 | |
| System Disease, | | | | CTSB | |
| Organismal Injury | | | | HSP90AA1 | |
| and Abnormalities | | | | JUND | |
| Liver lesion | | | | KLF6 | |
| | | | | LPP | |
| | | | | NEAT1 | |
| | | | | RBM47 | |
| | | | | RNFT2 | |
| | | | | SEC63 | |
| | | | | SLC35A3 | |
| | | | | SLC5A12 | |
| | | | | SRRM2 | |
| | | | | TRIM38 | |
| | | | | TTC39B | |
| | | | | ZNF740 | |
| Cellular Growth and | 0.00263 | | −0.2 | CTSB | 5 |
| Proliferation, | | | | JUND | |
| Connective Tissue | | | | KLF6 | |
| Development | | | | NEAT1 | |
| and Function, Tissue | | | | ZFP36L1 | |
| Development | | | | | |
| Proliferation of | | | | | |
| connective tissue cells | | | | | |
| Cellular Development, | 0.00343 | | −0.2 | CTSB | 5 |
| Cellular Growth and | | | | JUND | |
| Proliferation, Colony | | | | KLF6 | |
| formation of tumor cell | | | | NEAT1 | |
| lines | | | | PAX6 | |
| Cellular Movement, | 0.000117 | | −0.4 | CAMK2N1 | 11 |
| Cell movement of | | | | CTSB | |
| tumor cell lines | | | | HSP90AA1 | |
| | | | | JUND | |
| | | | | KLF6 | |
| | | | | LPP | |
| | | | | NEAT1 | |
| | | | | RALBP1 | |
| | | | | RBM47 | |
| | | | | SLC35A3 | |
| | | | | ZFP36L1 | |
| Cell Death and | 0.00136 | | −0.4 | CTSB | 5 |
| Survival, Organismal | | | | JUND | |
| Injury and | | | | KLF6 | |
| Abnormalities Cell | | | | NEAT1 | |
| death of connective | | | | RALBP1 | |
| tissue cells | | | | | |
| Organismal Injury and | 0.000167 | | −0.4 | RABL6 | 25 |
| Abnormalities | | | | RALBP1 | |
| Abdominal lesion | | | | RBM47 | |
| | | | | RNFT2 | |
| | | | | SEC63 | |
| | | | | SLC35A3 | |
| | | | | SLC5A12 | |
| | | | | SRRM2 | |
| | | | | TRIM38 | |
| | | | | TTC39B | |
| | | | | ZFP36L1 | |
| | | | | ZNF740 | |
| Cellular Movement | 0.000354 | | −0.5 | CAMK2N1 | 10 |
| Migration of tumor cell | | | | CTSB | |
| lines | | | | HSP90AA1, | |
| | | | | KLF6 | |
| | | | | LPP | |
| | | | | NEAT1 | |
| | | | | RALBP1 | |
| | | | | RBM47 | |
| | | | | SLC35A3 | |
| | | | | ZFP36L1 | |
| | | | | CHPT2P | |
| Cancer, Organismal | 0.00108 | | −0.5 | RALBP1 | 24 |
| Injury and | | | | RBM47 | |
| Abnormalities | | | | RNFT2 | |
| Intraabdominal organ | | | | SEC63 | |
| tumor | | | | SLC35A3 | |
| | | | | SLC5A12 | |
| | | | | SRRM2 | |
| | | | | TRIM38 | |
| | | | | TTC39B | |
| | | | | ZFP36L1 | |
| | | | | ZNF740 | |
| Cell Death and | 0.012 | | −0.6 | CTSB | 8 |
| Survival, Organismal | | | | HSP90AA1 | |
| Injury and | | | | JUND | |
| Abnormalities Cell | | | | KLF6 | |
| death of tumor cell | | | | NEAT1 | |
| lines | | | | PAX6 | |
| | | | | RABL6 | |
| | | | | RALBP1 | |
| Cell Death and | 0.0135 | | −0.8 | CTSB | 10 |
| Survival Apoptosis | | | | HSP90AA1 | |
| | | | | JUND | |
| | | | | KLF6 | |
| | | | | NEAT1 | |
| | | | | PAX6 | |
| | | | | RAB11B | |
| | | | | RABL6 | |
| | | | | RALBP1 | |
| | | | | ZFP36L1 | |
| Tissue Development | 0.00514 | | −1.1 | CTSB | 5 |
| Growth of epithelial | | | | JUND | |
| tissue | | | | PAX6 | |
| | | | | RBM47 | |
| | | | | ZFP36L1 | |
| Organismal Injury and | 0.019 | | −1.5 | CTSB | 10 |
| Abnormalities, | | | | HSP90AA1 | |
| Organismal Survival | | | | KLF6 | |
| Organismal death | | | | LPP | |
| | | | | NEAT1 | |
| | | | | RBM47 | |
| | | | | SEC63 | |
| | | | | SLC5A12 | |
| | | | | TRIM38 | |
| | | | | ZFP36L1 | |

The methods described herein can thus be used to evaluate complex, multivariate relationships between the host transcriptome and microbiome and significantly advance personalized medicine. These investigations and the directed treatments derived therefrom would not be feasible without the methods disclosed herein.

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of producing a sequencing library from human mRNA in a stool sample, the method comprising:
a) treating a human stool sample with a stool stabilizing reagent;
b) isolating RNA from the human stool sample treated with the stool stabilizing reagent based on the presence of a polyadenylated (polyA) tail;
c) obtaining a sample of isolated RNA comprising less than about 200 ng of polynucleotides; and
d) reverse transcribing the human mRNA using polyA-targeting primers and incorporating a unique molecular identifier into the reverse transcribed human mRNA.

2. The method of claim 1, wherein:
steps c)-d) are repeated to produce a plurality of sequencing libraries;
the stool stabilization reagent inhibits degradation of polynucleotides; or
the sample of the isolated RNA comprises less than about 175 ng, less than about 150 ng or less than about 125 ng of polynucleotides.

3. The method of claim 2, wherein:
the number of human genes detectable by the plurality of sequencing libraries relative to a single sequencing library is at least about 2-fold greater;
inhibiting degradation of polynucleotides comprises inhibiting DNase and RNase activity; or
the sample of the isolated RNA comprises about 100 ng of polynucleotides.

4. The method of claim 2, wherein steps c)-d) are repeated about two or more times, about three or more times, about four or more times, about five or more times, or about six or more times.

5. A method of detecting the presence of a human gene sequence expressed in a human gut, the method comprising:
e) sequencing the plurality of sequencing libraries produced by the method of claim 2; and
f) mapping the sequences produced in step e) using a computer algorithm.

6. The method of claim 5, wherein the computer algorithm comprises an adjusted mismatch penalty or an adjusted match bonus setting.

7. The method of claim 5, wherein said mapping results in an alignment rate of at least about 10%, at least about 20%, at least about 30%, at least about 40%, at least about 50%, or at least about 60%.

8. A sequencing library produced by the method of claim 1.

9. The sequencing library of claim 8, wherein the library comprises polynucleotide sequences complementary to at least about 3,000 human protein coding genes, at least about 4,000 human genes, at least about 5,000 human genes, or at least about 6,000 human genes.

10. The method of claim 1, wherein:
the human stool sample is from an infant; or
the human stool sample is collected following a dietary intervention.

11. The method of claim 10, wherein:
the infant is a preterm infant; or
the dietary intervention comprises administering human milk, infant formula, modified infant formula.

12. The method of claim 11, wherein the modified infant formula comprises bioactive proteins, bioactive fats, bioactive carbohydrates, prebiotics, fermentable substrates, human milk oligosaccharides, probiotics, live microbes, fecal microbial transplants, or a combination of any thereof.

13. The method of claim 1, wherein the human stool sample is collected following a medical intervention.

14. The method of claim 13, wherein the medical intervention comprises cesarean delivery, antibiotic administration, or intestinal surgery or resection, including for necrotizing enterocolitis.

15. The method of claim 14, wherein the intestinal surgery or resection comprises necrotizing enterocolitis.

16. A method of sequencing a nutrient absorption gene from an infant stool sample, the method comprising:
a) treating an infant stool sample with a stool stabilizing reagent;
b) isolating RNA from the infant stool sample treated with the stool stabilizing reagent based on the presence of a polyadenylated (polyA) tail;
c) obtaining a sample of isolated RNA comprising less than about 200 ng of polynucleotides;
d) reverse transcribing the human mRNA using polyA-targeting primers and incorporating a unique molecular identifier into the reverse transcribed human mRNA;
e) repeating steps c)-d) to produce a plurality of sequencing libraries; and
f) sequencing the plurality of sequencing libraries.

17. The method of claim 16, the method further comprising:
mapping the sequences produced in step f) using a computer algorithm; and detecting the presence of a decreased or increased level of a nutrient absorption gene mRNA relative to a control; or
detecting the presence of an increased level of SLC1A1, SLC38A1, SLC38A2, ABCG5, SLC26A2, LPL, SAR1B, SLC44A1, or BTD mRNA relative to an appropriate control.

18. The method of claim 16, the method further comprising mapping the sequences produced in step f) using a computer algorithm; and detecting the presence of a decreased or increased level of a nutrient absorption gene, a nutrient transporter gene, barrier function gene, a hypoxia-related gene, a GI ischemia-related gene, a heat shock protein gene, an HDAC response gene, a butyrate metabolism gene, an energy utilization gene, a stemness gene, or an immune response gene mRNA relative to a control.

19. A method of treating an infant with feeding intolerance comprising:
a) obtaining a sample from the infant that comprises at least a first mRNA associated with feeding intolerance;
b) detecting in the sample the presence of an increased or decreased level of the mRNA relative to a control infant that lacks feeding intolerance; and
c) treating the infant with feeding intolerance based on the increased or decreased level of the mRNA relative to the control.

20. The method of claim 19, wherein treating the infant comprises:
no treatment;
administering intravenous nutrition; or
administering a partially hydrolyzed formula or human milk treated with enzymes.
